(19) United States
(12) Patent Application Publication
(10) Pub. No.: US 2023/0320611 A1
(43) Pub. Date: Oct. 12, 2023

(54) SYSTEM AND METHOD OF ACCURATE QUANTITATIVE MAPPING OF BIOPHYSICAL PARAMETERS FROM MRI DATA

(71) Applicant: Cornell University, Ithaca, NY (US)

(72) Inventors: Yi Wang, New York, NY (US); Yan Wen, New York, NY (US); Ramin Jafari, New York, NY (US); Thanh Nguyen, Forest Hills, NY (US); Pascal Spincemaille, New York, NY (US); Junghun Cho, New York, NY (US); Qihao Zhang, New York, NY (US)

(73) Assignee: Cornell University, Ithaca, NY (US)

(21) Appl. No.: 18/042,163

(22) PCT Filed: Aug. 19, 2021
(86) PCT No.: PCT/US2021/046744

§ 371 Date: Feb. 17, 2023

Related U.S. Application Data
(60) Provisional application No. 63/068,228, filed on Aug. 20, 2020.

(51) Int. Cl. A61B 5/055; A61B 5/00

(52) U.S. Cl. CPC A61B 5/055; A61B 5/0042; A61B 5/4872; G06T 7/00

(57) ABSTRACT

Quantitative susceptibility mapping methods, systems and computer-accessible medium generate images of tissue magnetism property from complex magnetic resonance imaging data using the Bayesian inference approach, which minimizes a cost function comprising of a data fidelity term and regularization terms. The data fidelity term is constructed directly from the multiecho complex magnetic resonance imaging data. The regularization terms include a prior constructed from matching structures or information content in known morphology, and a prior constructed from regions of low susceptibility contrasts characterized on image features. The quantitative susceptibility map can be determined by minimizing the cost function that involves nonlinear functions in modeling the obtained signals, and the corresponding inverse problem is solved using nonconvex optimization using a scaling approach or deep neural network. The nonconvex optimization is also developed for solving other inverse problems of nonlinear signal models in fat-water separation, tissue transport and oxygen extraction fraction.

### CROSS REFERENCE TO RELATED APPLICATIONS

This application claims priority to U.S. Provisional Patent Application Ser. No. 63/068,228, titled “SYSTEM AND METHOD OF ACCURATE QUANTITATIVE MAPPING OF BIOPHYSICAL PARAMETERS FROM MRI DATA,” filed on Aug. 20, 2020, said application being incorporated by reference herein in its entirety.

### STATEMENT REGARDING FEDERALLY SPONSORED RESEARCH AND DEVELOPMENT

This invention was made with government support under Grant No. CA181566, DK116126 NS090464, NS095562 and NS105114 awarded by the National Institutes of Health. The government has certain rights in this invention.

### TECHNICAL FIELD

This invention relates to magnetic resonance imaging, and more particularly to quantitatively mapping material intrinsic physical properties using signals collected in magnetic resonance imaging.

### BACKGROUND

Quantitative susceptibility mapping (QSM) in magnetic resonance imaging (MRI) has received increasing clinical and scientific interest. QSM is useful for characterizing and quantifying magnetic chemical compositions such as iron, calcium, and contrast agents including gadolinium and superparamagnetic iron oxide nanoparticles. Tissue composition of these compounds may be altered in various neurological disorders, such as Parkinson's disease, Alzheimer's disease, stroke, multiple sclerosis, hemochromatosis, and tumor as well as other diseases throughout the body. Different from diffuse tissue iron, deoxyheme iron in the capillaries and veins reflects tissue metabolic oxygen extraction from circulation, and circulation is vital to all organs and is perturbed in many diseases. QSM is able to unravel novel information related to the magnetic susceptibility (or simply referred as susceptibility), a physical property of underlying tissue that depends on magnetic chemical compositions. Due to the prevalence of iron and calcium in living organisms and their active involvement in vital cellular functions, QSM is in general very useful to study the molecular biology of iron/calcium by tracking iron/calcium in the circulation and other body system and to study the metabolic activities by using iron and calcium as surrogate marks. QSM can also be used to quantify contrast agents passage in tissue, which can be captured in time resolved imaging and fitted to physical transport equations for generating quantitative maps of transport parameters. Therefore, accurate mapping of the magnetic susceptibility induced by iron, calcium and contrast agents will provide tremendous benefit to clinical researchers to probe the structure and functions of human body, and to clinicians to better diagnose various diseases and provide relevant treatment.

QSM reconstruction has been based on minimizing a cost function that fits the proton-experienced tissue magnetic field b(r) (in unit B0, main field of MRI scanner) to the unit dipole field

$$d ( r ) = \frac{2 {z}^{2} - {x}^{2} - {y}^{2}}{4 π {\vert r \vert}^{5}} .$$

Approximating noise in b as Gaussian, the data fidelity term to fit measured data with biophysics model is [symbol] w(r)|d*χ(r)−b(r)|²dr≡∥d*χ−b∥_{w}². This integral form b=d*χ allows numerical estimation of the susceptibility solution, typically under a regularization associated with a prior information. As known from physics, the integral form represents a solution to the governing partial differential equation (PDE). For mathematical analysis of susceptibility solution, the equivalent PDE that can be derived directly from Maxwell's equation with Lorentz sphere correction to connect the macroscopic magnetic susceptibility quantity x to the microscopic field b experienced by protons, signal generators in MRI:

$$( {\partial }_{z}^{2} - ( {\partial }_{x}^{2} + {\partial }_{y}^{2} ) \frac{1}{2} ) χ = - \frac{3}{2} Δ b . \qquad [ 1 ]$$

Eq. 1 is a wave equation with respect to susceptibility χ (z-axis is time) and a Laplacian equation with respect to field b. In the continuous space without any error, b(r)=d*χ(r), B(k)=D(k)X(k), allows a precise solution of no streaking:

$$χ = {lim}_{ϵ ↘ 0} {- 1}^{} ( ( 1 - {η}_{ϵ} ) ( B / D ) ) \cdot {η}_{ϵ} = 1 if D ( k ) < ϵ / 2 otherwise 0.$$

Any field data that cannot be fitted to the dipole field is referred as the incompatible field data v(r), which may come from various sources including noise, digitiztion error, and anisotropy source. This incompatible field causes artifacts according to the wave propagation solution:

$${χ}_{v} ( r ) = \int \frac{- \frac{3}{2} {Δ}^{′} v ( {r}^{′} )}{2 π \sqrt{{( z - {z}^{′} )}^{2} - 2 ( {( x - {x}^{′} )}^{2} + {( y - {y}^{′} )}^{2} )}} {dr}^{′} , \qquad [ 2 ]$$

$${( z - {z}^{′} )}^{2} > 2 ( {( x - {x}^{′} )}^{2} + {( y - {y}^{′} )}^{2} ) .$$

### Here

$$\frac{1}{2 π \sqrt{{( z - {z}^{′} )}^{2} - 2 ( {( x - {x}^{′} )}^{2} + {( y - {y}^{′} )}^{2} )}}$$

is the wave propagator having large values at magic-angle cones above and below the incompatible source

$$- \frac{3}{2} {Δ}^{′} v ( {r}^{′} ) .$$

The dipole incompatible part, including noise, discretization error, and anisotropy sources, generates artifacts defined by the wave propagator with the B0 field direction as the time axis. Granular noise causes streaking along wave propagator cone surface and contiguous error causes both streaking and shadowing artifacts. QSM reconstruction task is to minimize these streaking and shadowing artifacts. The streaking can be effectively minimized by penalizing edges that are not in tissue edges, which is typically referred as a structure prior. The shadowing artifacts can also be suppressed but remain challenging.

The multiecho gradient echo (mGRE) sequence is widely accessible and commonly used to acquire data for QSM. Prior QSM reconstruction has been based on the approach of estimating the magnetic field from all echo signals of the mGRE data before the field to magnetic susceptibility inversion. This estimating the field from all echoes in prior art has invariably involved approximations in noise characteristics, violating fidelity to mGRE data at all echo times and resulting in errors.

In general, magnetic susceptibility affects the complex MRI signal in a nonlinear manner. Further biophysical decomposition of susceptibility contributors, including fat with chemical shift, deoxyheme iron vs other diffuse ferritin iron, and contrast agents and of tissue transport and interaction phenomenon add complexity to the nonlinear signal models. The corresponding inverse problems of nonlinear signal models are nonconvex and their solutions challengingly depend on initialization.

### SUMMARY

Implementations of systems and methods for collecting and processing multiecho (including the single echo as a special case) complex MRI signals of a subject, and reconstructing maps of physical properties intrinsic to the subject (e.g., magnetic susceptibility) are described below. In some implementations, MR signal data corresponding to a subject can be transformed into susceptibility-based images that quantitatively depict the structure and/or composition and/or function of the subject. Using this quantitative susceptibility map, one or more susceptibility-based images of the subject can be generated and displayed to a user. The user can then use these images for diagnostic or experimental purposes, for example to investigate the structure and/or composition and/or function of the subject, and/or to diagnose and/or treat various conditions or diseases based, at least in part, on the images. As one or more of the described implementations can result in susceptibility-based images having higher quality and/or accuracy compared to other susceptibility mapping techniques, at least some of these implementations can be used to improve a user's understanding of a subject's structure and/or composition and/or function, and can be used to improve the accuracy of any resulting medical diagnoses or experimental analyses.

Some of the implementations described below for the magnetic dipole inversion using incorporation of multiecho complex data based cost function, preconditioning, deep neural network, chemical composition based fat compensation, and enforcement of regional susceptibility contrasts improve susceptibility map quality including suppression of artificial signal variation in regions of low susceptibility contrasts and to provide accurate susceptibility values with precise fidelity to the acquired multiecho complex MRI data, which are pressing concerns to ensure quantification accuracy, experimental repeatability and reproducibility.

In general, one aspect of the invention disclosed here enables an accurate quantification of tissue magnetic susceptibility by the use of receiving multiecho complex magnetic resonance imaging data obtained by a magnetic resonance scanner, wherein the multiecho data at each echo time is complex and comprises magnitude and phase information or real and imaginary information regarding the object; and by the use of estimating a magnetic susceptibility distribution of the object based on the multiecho complex data at each echo time, wherein estimating the magnetic susceptibility distribution of the subject comprises determining a cost function, the cost function relating at least a data fidelity term involving a comparison between the measured and a susceptibility modeled complex signal at each echo time wherein the susceptibility modeled complex signal has time dependence in both signal magnitude and phase, and a regularization term associated with a structure prior information, and determining a magnetic susceptibility distribution based on minimizing the cost function

In general, another aspect of the invention disclosed here enables accurately estimating the magnetic susceptibility distribution, including calculating a magnetic field experienced by the tissue in the object based on modeling the multiecho complex data with consideration of noise in both magnitude and phase information at each echo time, and replacing the temporal phase evolution with echo time in multiecho data with a phase factor determined by the calculated magnetic field.

In general, another aspect of the invention disclosed here for quantitative susceptibility mapping introduces a scaling method, wherein the magnetic field is divided first by a factor for estimating the magnetic susceptibility and then the output magnetic susceptibility is multiplied by the factor.

In general, in another aspect of the invention disclosed here for quantitative susceptibility mapping in the body comprised of fat and water, the magnetic field, the water fraction and the fat fraction in a voxel are computed using a deep neural network.

In general, another aspect of the invention disclosed here for estimating the magnetic susceptibility distribution includes identifying regions distributed over the whole image volume that have low susceptibility contrast according to a characteristic feature and adding a second regularization term associated with penalizing susceptibility variations in these regions. This regularization over low contrast regions allows for estimation and reduction of shadowing and/or streaking artifacts in these regions.

In general, another aspect of the invention disclosed here for estimating the magnetic susceptibility distribution includes using a characteristic feature characterized by R2* value. The region of interest is determined by similar R2* properties of contiguous voxels; and the penalty of the second regularization term depends on a representative R2* value in the region of interest.

In general, in another aspect of the invention disclosed here for estimating the magnetic susceptibility distribution, the cost function minimization involves preconditioning.

In general, another aspect of the invention disclosed here for estimating the magnetic susceptibility distribution is realized using a deep neural network.

Implementations of these aspects may include one or more of the following features.

In some implementations, the cerebrospinal fluid in the ventricles in the brain is segmented based on low R2* values according to similar R2* properties of contiguous voxels.

In some implementations, the prior information regarding the magnetic susceptibility distribution is determined based on an L1 norm and structural information of the object estimated from acquired images of the object.

In some implementations, the object comprises a cortex, a putamen, a globus pallidus, a red nucleus, a substantia nigra, or a subthalamic nucleus of a brain, or a liver in an abdomen, and generating one or more images of the object comprises generating one or more images depicting the cortex, the putamen, the globus pallidus, the red nucleus, the substantia nigra, or the subthalamic nucleus of the brain, or the liver in the abdomen.

In some implementations, the object comprises at least one of a multiple sclerosis lesion, a cancerous lesion, a calcified lesion, or a hemorrhage, and generating one or more images of the object comprises generating one or more images depicting at least one of the multiple sclerosis lesion, the cancerous lesion, the calcified lesion, or the hemorrhage.

In some implementations, the operations further comprise quantifying, based on the one or more images, a distribution of contrast agents introduced into the object in the course of a contrast enhanced magnetic resonance imaging procedure. The contrast agent passage through tissue is modeled according to a flow velocity and exchange coefficients. The contrast agent passage is captured on many timeframes of time resolved imaging and is used to determined velocity and exchange coefficients by solving the inverse problem of transport equation.

In some implementations, the operations further comprise quantifying, based on the one or more images, a distribution of fat and water fractions. The fat chemical shift effect on signal phase is captured on multiecho magnetic resonance imaging data and is used to generate fat and water decomposition, which is a nonconvex optimization problem that can be solved using a deep neural network without training of labeled data.

In some implementations, the operations further comprise quantifying, based on the one or more images, a distribution of oxygen extraction fraction. The deoxyheme iron has unique effect on multiecho magnetic resonance imaging data and is used to separate deoxygeme iron from other diffuse susceptibility sources, which is a nonconvex optimization problem that is solved using a deep neural network without training of labeled data.

In some examples, a method for generating one or more images of an object comprises obtaining complex magnetic resonance data collected by a magnetic resonance scanner, wherein the data is complex and comprises magnitude and phase information regarding the object; estimating a magnetic susceptibility distribution of the object based on the obtained complex magnetic resonance data, wherein estimating the magnetic susceptibility distribution of the subject comprises: determining a cost function, the cost function including a data fidelity term involving modeling a magnetic susceptibility effect on the obtained complex magnetic resonance data, a regularization term involving a first region with a low feature of susceptibility contrasts, and a second region with a feature of susceptibility contrasts different from the feature of susceptibility contrasts of the first region; minimizing the cost function; determining a magnetic susceptibility distribution based on the act of minimizing the cost function; and presenting, on a display device, one or more images of the object generated based on the determined magnetic susceptibility distribution. In some examples, the aforementioned example method may include one or more of the following particulars, singly or in any combination: the regularization term involves a third and/or more regions with features of susceptibility contrasts differing from features of susceptibility contrasts of the first two regions; the feature of susceptibility contrasts of the first region is the lowest; the regularization term involves a penalty on a variation of susceptibility values in a region; the penalty varies with the feature of susceptibility contrasts of a region; the feature of susceptibility contrasts of a region involves decay rates of magnitude signals in voxels of the region; a region is formed by binning a group of voxels sharing a characteristic; the characteristic is a distribution of decay rates; the distribution is a rectangular or Gaussian function; the characteristic is a distribution in space; minimizing the cost function is obtained using a preconditioning method; the regularization term is implicitly formed and estimating the magnetic susceptibility distribution is realized using a deep neural network; the deep neural network is trained with labeled data or unlabeled data, or is untrained; the deep neural network is trained with network weights updated with a test data; modeling a magnetic susceptibility effect on the obtained complex magnetic resonance data involves a comparison between the obtained complex magnetic resonance data and a susceptibility modeled complex signal at each echo time; the susceptibility modeled complex signal includes a phase involving echo time and the magnetic susceptibility dipole convolution; the comparison comprises an Lp norm of the difference between the obtained complex magnetic resonance data and a susceptibility modeled complex signal at each echo time; modeling a magnetic susceptibility effect on the obtained complex magnetic resonance data comprises calculating a magnetic field experienced by tissue in the object from the obtained magnetic resonance data; calculating a phase at each echo time according to the calculated magnetic field and a multiplicative scaling factor; performing a comparison involving the calculated phase with a phase of the magnetic susceptibility dipole convolution at each echo time; and dividing the determined magnetic susceptibility distribution by the scaling factor; the comparison involving the calculated phase with a phase of the magnetic susceptibility dipole convolution at each echo time comprises calculating an Lp norm of a weighted difference between an exponential factor of the calculated phase and an exponential factor of the phase of the magnetic susceptibility dipole convolution at each echo time.

In some examples, a method for generating one or more images of an object comprises obtaining multiecho complex magnetic resonance data collected by a magnetic resonance scanner, wherein the multiecho data is complex and comprises magnitude and phase information regarding the object; determining oxygen extraction fraction distribution of the object based on the multiecho complex magnetic resonance data, comprising: determining a cost function, the cost function including a data fidelity term involving a comparison between the obtained multiecho complex magnetic resonance data and a modeled complex signal at each echo time with a consideration of deoxyheme-iron effects on magnitude and phase of the modeled complex signal, and a regularization term; minimizing the cost function; determining an oxygen extraction fraction distribution based on the act of minimizing the cost function; and presenting, on a display device, one or more images of the object generated based on the determined oxygen extraction fraction distribution. In some examples, the aforementioned example method may include one or more of the following particulars, singly or in any combination: the consideration of deoxyheme-iron effects on signal magnitude and phase includes a model of deoxyheme-iron distributed in cylinders and nonblood susceptibility sources distributed diffusely in space for determining the modeled complex signal; the comparison between the obtained multiecho complex magnetic resonance data and the modeled complex signal comprises an Lp norm of the difference of the obtained multiecho complex magnetic resonance data and the modeled complex signal. In some examples, the one or more images of the object include the nonblood susceptibility distribution and/or venous blood volume; wherein the regularization term includes a sparsity of edges in oxygenation and/or venous blood volume; wherein minimizing the cost function includes a dipole deconvolution step and the regularization term includes a sparsity of edges in tissue susceptibility and/or penalties on variations of susceptibility values in regions; wherein the regularization term includes a sparsity of temporal evolution along echo time of the magnitude of the modeled complex signal; wherein minimizing the cost function includes coordinate descent along axes defined by variables of oxygenation and venous blood volume; wherein the regularization term is implicitly formed and determining the oxygen extraction fraction distribution is realized using a deep neural network; wherein the deep neural network is trained with labeled data or unlabeled data, or is untrained; wherein the deep neural network is trained with network weights updated with a test data.

In some examples, a method for generating one or more images of an object comprises obtaining multiple timeframe imaging data about tracer transport through the object; determining tracer concentration at each timeframe from imaging data according to tracer effects on image signal; estimating tracer velocity and exchange coefficient distribution of the object, comprising: determining a cost function, the cost function including a data fidelity term involving a comparison between the determined tracer concentrations and tracer concentrations according to a velocity-exchange model; and minimizing the cost function; and determining a velocity and exchange distribution based on the act of minimizing the cost function; and presenting, on a display device, one or more images of the object generated based on the determined velocity and/or exchange distribution. In some examples, the aforementioned example method may include one or more of the following particulars, singly or in any combination: the multiple timeframe imaging data is obtained using magnetic resonance imaging; the multiple timeframe imaging data is obtained using positron emission tomography; the multiple timeframe imaging data is obtained using computed tomography, or single photon emission tomography; the velocity-exchange model includes a temporal deconvolution involving an exchange coefficient; the comparison between the obtained tracer concentrations and tracer concentrations according to a velocity-exchange model comprises an Lp norm of a difference between the obtained tracer concentrations and tracer concentrations according to a velocity-exchange model; the cost function includes a regularization term imposing a sparsity on velocity and/or exchange coefficient distribution; the regularization term is implicitly formed and determining the velocity and exchange coefficient distribution is realized using a deep neural network; the deep neural network is trained with labeled data or unlabeled data, or is untrained; the deep neural network is trained with network weights updated with a test data.

In some examples, a method for generating one or more images of an object comprises obtaining multiecho complex magnetic resonance imaging data collected by a magnetic resonance scanner, wherein the multiecho data is complex and comprises magnitude and phase information regarding the object; estimating fat and water distribution of the object based on the multiecho complex data, comprising: determining a cost function, the cost function including a data fidelity term involving a comparison between the obtained multiecho complex magnetic resonance data and a complex signal of a fat-water model at each echo time wherein the fat-water model has echo time dependence in both signal magnitude and phase with the fat chemical shift contributing an additional phase; and determining fat and water distribution of the object using a deep neural network without labeled data; and presenting, on a display device, one or more images of the object generated based on the determined fat and water distribution. In some examples, the aforementioned example method may include one or more of the following particulars, singly or in any combination: the comparison between the obtained multiecho complex magnetic resonance data and a complex signal of a fat-water model at each echo time comprises an Lp norm of a difference between the obtained multiecho complex magnetic resonance data and a complex signal of a fat-water model at each echo time; the fat-water model has echo time dependence in signal magnitude with different decay rates for fat and water components; the fat chemical shift contributing an additional phase comprises a sum of contributions from a fat chemical shift spectrum; the deep neural network is trained with unlabeled data; network weights of the trained deep neural network are updated with a test data; the deep neural network is untrained; the deep neural network includes a U-net.

Details of one or more embodiments are set forth in the accompanying drawings and the description below. Other features and advantages will be apparent from the description and drawings, and from the claims.

### BRIEF DESCRIPTION OF THE DRAWINGS

FIGS. 1a-1b show a comparison between Total Field Inversion (TFI), TFI+model error reduction through iterative tuning (MERIT), and Multiecho Complex Total Field Inversion (mcTFI) in a simulated brain that contains a calcified lesion and an intracerebral hemorrhage (ICH). a) TFI contains streaking artifacts that originated from the two lesions, due to improper noise weighting in voxels around the lesions. MERIT helped to suppress the streaking artifacts, but the size of the lesion appeared enlarged. mcTFI suppressed the streaking artifacts and reconstructed the lesions best resembled the ground truth. b) mcTFI results also have the best root-mean-square error (RMSE) at all different SNR levels.

FIGS. 2a-2b show a comparison between TFI, TFI+MERIT, and mcTFI in one of the datasets provided by the QSM Challenge 2.0. a) TFI contains streaking artifacts that originated from the calcification, whereas both TFI+MERIT and mcTFI were able to suppress the streaking artifacts. b) mcTFI demonstrated a more accurate reconstruction (last two columns) of the calcification than TFI+MERIT (bold indicate the best value).

FIG. 3 shows QSM reconstructions in a brain with the large ICH. Severe streaking artifacts were observed in the TFI result. TFI+MERIT reduced the severity of the streaking artifacts but did not completely suppress them (arrows), likely because MERIT did not properly penalize all of the problematic voxels. The streaking artifacts were the smallest in the mcTFI reconstructed susceptibility map.

FIGS. 4a-4c shows mcTFI QSM reconstruction with and without shadowing artifacts reduction (SAR). a) the masks of the first three bins represent cerebrospinal fluid, cortical gray and white matter, respectively. b) axial and c) sagittal view of shadowing artifacts (arrows, left images) and their reductions (SAR, right images)

FIG. 5 shows deep network architecture, input complex signal magnitude and phase (|S|, φ(S)), intermediate layers output images, and network outputs including magnitude and phase of water(|W|, φ(W)), magnitude and phase of fat (|F|, φ(F)), field (f), and R*₂ maps are shown for a test dataset after training was completed.

Part a) of FIG. 6 shows water, fat, field and R2* reference images in a healthy volunteer. Parts b), c), and d) of FIG. 6 show corresponding results for supervised (STD), unsupervised (UTD), and no-training (NTD) methods. Region of interest (ROI) measurement linear regression analysis showed excellent agreement between each deep neural network (DNN) method and the reference T2*-IDEAL reconstruction for proton density fat fraction, field, and R2* with R²≥0.98 and slope ∈[0.93, 1.05].

Part a) of FIG. 7 shows water, fat, field and R2* reference images are shown in a moderate iron-overload patient. Parts b), c), and d) of FIG. 7 show corresponding results for supervised (STD), unsupervised (UTD), and NTD methods. ROI measurement linear regression analysis shows excellent agreement between each DNN method and the reference T2*-IDEAL reconstruction for proton density fat fraction, field, and R2* with R²≥0.96 and slope ∈[0.93, 1.03].

FIG. 8 shows training and validation loss results for supervised (STD) and unsupervised (UTD) training methods.

FIG. 9A depicts water/fat separation results comparing current T2*-IDEAL without (1) and with (2) initialization with the proposed NTD method (3) training results for field (a), water (b), R2* (c), and fat (d). No initial guess was used for water and fat maps.

FIG. 9B depicts corresponding loss curves (e) are shown for T2*-IDEAL Vs. iteration and NTD Vs. epoch.

FIGS. 10a-10d show quantitative transport mapping in the livers with cancers. a) time-resolved dynamic contrast enhanced MRI (7 timeframes) and c) corresponding velocity mapping for a liver with focal nodular hyperplasia (FNH). b) time-resolved dynamic contrast enhanced MRI (7 timeframes) and d) corresponding velocity mapping for a liver with hepatocellular carcinoma (HCC). The benign FNH lesion has lower velocity (arrow in c), while the malignant HCC lesion has higher velocity (arrow in d).

FIG. 11 shows a brain with ischemic lesions. The lesion prominent on diffusion weighted imaging (DWI, white arrows) has a slightly elevated oxygen extraction fraction (OEF), indicating viable. The lesion prominent on T2 FLAIR (fluid attenuated inversion recovery, black arrows) has diminished OEF, indicating nonviable and potential hemorrhagic transformation risk for revascularization.

FIGS. 12a-12b show example processes for mapping tissue magnetic susceptibility a) using the magnetic field estimation and b) without using the magnetic field estimation.

FIG. 13 is a block diagram of an example computer system.

FIG. 14 depicts a 3D U-net structure implemented in the experiment.

Parts (a)-(b) of FIG. 15 are simulated artery and vein vasculature in an 8 mm³ cube. Parts (c)-(f) of FIG. 15 are simulated F, K^{trans}, Vₚ and Vₑ maps, respectively. Parts (a)-(d) of FIG. 16 are reconstructed F, K^{trans}, Vₚ and Vₑ maps (left, respectively) comparing to ground truth (right, respectively), in a validation dataset.

Parts (a)-(d) of FIG. 17 are reconstructed F, K^{trans},Vₚ, and Vₑ maps (left, respectively) comparing to ground truth (right, respectively) in simulation with real tumor vasculature.

Part (a) of FIG. 18 shows post contrast of a dynamic contrast-enhanced (DCE) MRI image of glioblastoma. Parts (b)-(e) of FIG. 18 are reconstructed F, K^{trans}, Vₚ and Vₑ maps, respectively, using a deep learning method.

Like reference symbols in the various drawings indicate like elements.

### DETAILED DESCRIPTION

Implementations of systems and methods for collecting and processing MRI signals of a subject, and reconstructing maps of physical properties intrinsic to the subject (e.g., magnetic susceptibility) are described below. Some of the disclosed implementations overcome limitations in quantitative susceptibility mapping (QSM), where the computation robustness and accuracy and the Bayesian prior information affect QSM image quality and practical usage. In some implementations, to reconstruct maps of a subject's magnetic susceptibility, the computation process of finding the susceptibility distribution that optimally fits available multiecho gradient echo MRI (mGRE) data and tissue structure prior information is made accurate by fitting data at each echo time without compromising the precision in modeling data noise. In some implementations, the computation process of finding the magnetic field from mGRE data is also made accurate by fitting data at each echo time without compromising the precision in modeling data noise. In some implementations, a scaling factor is used to ensure robust initialization of the nonconvex optimization needed to deal with precise signal noise modeling. In some implementations, additional information of regions with low susceptibility contrasts that can be automatically segmented from R2* map is used to improve QSM reconstruction. In some implementations, a deep neural network is used for magnetic field estimation and QSM reconstruction. Implementations of these improvements have made QSM technology robust and accurate.

For multiecho gradient echo (mGRE) magnetic resonance imaging data obtained by a magnetic resonance scanner, the mGRE data at each echo time is complex, its magnitude and phase information or real and imaginary information regarding the object is well characterized to have Gaussian noise in the domain of complex signal. This mGRE data should be generally interpreted as multiple echoes with both magnitude and phase evolving with echo times. Gradient echo, spin echo and hybrid echo and other type of pulse sequences can be used to acquire these multiple echoes. Therefore, these data may be referred to as multiecho complex (MC) data.

QSM prior art typically consists of (1) estimating the magnetic field from multiecho complex signals at all echo times and 2) performing the inversion from magnetic field to magnetic susceptibility. The first step here typically focuses on signal phase, ignoring noise effects on signal magnitude; the second step here invariably has difficulties with the very complex noise property of the estimated field. Consequently, both steps contribute errors to QSM reconstruction.

To avoid these two steps and their associated errors, our invention improves upon QSM prior art through estimating a magnetic susceptibility distribution of the object based on the multiecho complex data at each echo time. We construct a cost function relating a data fidelity term associated with a likelihood function of the Gaussian noise in the multiecho complex data at each echo time. The likelihood function for the multiecho complex data at an echo time is Gaussian with the variance same for all echoes but the mean depending on individual signals. The product of likelihood functions of all echoes for the resultant likelihood function. The negative log of the likelihood function is the cost function referred to as a data fidelity term, and the maximal likelihood estimation corresponds to the minimal cost function. This leads to that the cost function for the QSM reconstruction is a sum of cost functions of all individual echoes. Extending to the Bayesian inference framework, the prior probability is the exponential function of the negative regularization term, the prior probability times the likelihood function determines the posterior probability, and the maximum a posterior probability (known as MAP) estimation leads to minimum of a cost function comprised of the data fidelity term from likelihood and the regularization term from prior probability. The data fidelity term involves summing a comparison between the measured signal and modeled signal at each echo. This comparison can be a difference expressed in an Lp norm (p≥0, for example, p=2 for Gaussian noise model). The modeled signal involves temporal evolution in both magnitude and phase. As the field to susceptibility inverse problem is ill-posed, additional information is needed for QSM reconstruction, which is a regularization term associated with a structure prior information. Then the magnetic susceptibility distribution is determined based on minimizing the cost function.

We extend the approach of going back to individual echo data to addressing errors in the first and second steps of QSM prior art. Therefore, the first step is improved by calculating a magnetic field experienced by the tissue in the object based on modeling the multiecho complex data with consideration of noise in both magnitude and phase information at each echo time, which would allow precise modeling noise in both magnitude and phase data. The second step is improved by replacing the temporal phase evolution with echo time in the multiecho complex data with a phase factor determined by the calculated magnetic field, which would allow proper estimation of noise weighting in formulating the data fidelity term. This is a nonlinear nonconvex optimization problem, which depends on initialization or may be trapped in local minima.

We devised a scaling method for solving nonlinear nonconvex optimization problem without concerns in initialization trapping. We scale the magnetic field down to be very small, which alleviates initialization problems and allows linearization of phase factors. The magnetic field is divided first by a factor for estimating the magnetic susceptibility. The small susceptibility values can be reliably searched. Then, the output magnetic susceptibility is multiplied by the factor, to obtain the properly values susceptibility distribution of the object.

For tissue with fat, we solve the fat water separation problem for obtaining the magnetic field in the presence of phase and magnitude effects from chemical shifts of fat and other compounds. This fat-water separation problem is a nonlinear nonconvex optimization problem that is dependent on initialization. We devised a deep neural network approach with or without training (supervised or unsupervised training) to overcome this initialization problem. The magnetic field, the water fraction and the fat fraction in a voxel can be computed using a deep neural network.

We further improve the use of structure prior information for effective suppression of artifacts in QSM reconstruction. The concept of penalizing susceptibility variation in a region of uniform susceptibility or zero susceptibility contrast can be extended to include regions of small susceptibility contrasts at graded penalties. A region with a susceptibility contrast slightly larger than zero can be characterized by a slightly lower penalty in the cost function. In this manner, estimating the magnetic susceptibility distribution include steps of identifying a region of interest in a tissue that has a low susceptibility contrast according to a characteristic feature and adding a second regularization term associated with enforcing the known low susceptibility contrast in the region of interest in the tissue. We use R2* values as the characteristic feature for identifying regions at various susceptibility contrasts. R2* values can be readily obtained from multiecho complex data. The region of interest at a given susceptibility contrast can be determined by similar R2* properties of contiguous voxels, and the penalty there depends on a representative R2* value in the region of interest.

We have developed several solver methods to perform the cost function minimization. One solver involves preconditioning that uses small search steps in low susceptibility regions and large search steps in high susceptibility regions (absolute value of susceptibility and water is zero-reference). Another solver for estimating the magnetic susceptibility distribution is realized using a deep neural network (DNN). The DNN can be trained under supervision of labeled data, including data generated from known biophysics models. The DNN can be trained with unlabeled data according to biophysics model of signal. The DNN can also be used without training to process a test data by minimizing a cost function based on the biophysics model of signal.

We have developed methods to improve MRI signal in regions with large R2* by acquiring zero or ultrashort echo time data immediately after RF excitation. Only a portion of k-space is acquired, and the undersampled data is reconstructed using structure consistency among images of various echo times, as expressed in sparsity or as learned by a deep neural network. This structure consistency is also used to shorten QSM data acquisition by undersampling data acquisition for all echo times.

Accurate QSM improves many applications, including mapping brain QSM with reduced shadowing and streaking artifacts, particularly in brains with large susceptibility contrasts such as from hemorrhages and tumor with angiogenesis blood leaks, mapping susceptibility sources in vessel wall plaque for differentiating intraplaque hemorrhage from plaque calcification, and mapping tissue iron in the livers of patients under blood transfusion treatment. Accurate QSM will also improve mapping of tissue oxygen extraction fraction and fat water fraction map. Various aspects of our invention are exemplified in the specific embodiments in the following implementations of systems and methods for collecting and processing MRI signals of a subject, and reconstructing maps of physical properties intrinsic to the subject.

In general, one implementation obtains complex magnetic resonance (MR) data collected by a magnetic resonance scanner, wherein the data is complex and comprises magnitude and phase information regarding the object. The magnetic susceptibility distribution of the object is estimated from the obtained complex MR data by determining a cost function. As the magnetic susceptibility affects MRI data through dipole convolution generated magnetic field, the cost function is designed to include a data fidelity term that involves the magnetic susceptibility dipole convolution modeling of the data phase. An advantage in reconstructing the magnetic susceptibility distribution is to reduce artifacts such as those described in the above Eq. 2, which streak and shadow throughout the whole image volume and can be reduced in low susceptibility-contrast regions that are distributed throughout the image volume. A feature is used to characterize the susceptibility contrast, such as R2* values. A first region of the lowest feature of the susceptibility contrast is cerebrospinal fluid distributed in the brain ventricles and sulci. A second region of the second lowest feature of the susceptibility contrast is the cortical gray matter adjacent to the cerebrospinal fluid. Additional regions may be considered, such as a third region of the third lowest feature of susceptibility contrast is the white matter adjacent to the cortical gray matter. Accordingly, the cost function is designed to include a regularization term involving two or more regions of the lowest features of susceptibility contrasts. The susceptibility is determined by minimizing the cost function. The regularization may include penalties on susceptibility variation in the regions of lowest features of susceptibility contrasts. The regularization may be formulated implicitly through a deep neural network.

In general, an example embodiment provides a method for generating one or more images of an object, the method comprising obtaining complex magnetic resonance data collected by a magnetic resonance scanner, wherein the data is complex and comprises magnitude and phase information regarding the object, estimating a magnetic susceptibility distribution of the object based on the obtained complex magnetic resonance data, wherein estimating the magnetic susceptibility distribution of the subject comprises determining a cost function, the cost function including a data fidelity term involving modeling a magnetic susceptibility effect on complex magnetic resonance data, a regularization term involving a first region with a low feature of susceptibility contrasts, and a second region with a feature of susceptibility contrasts different from the feature of susceptibility contrasts of the first region; minimizing the cost function, determining a magnetic susceptibility distribution based on the act of minimizing the cost function, and presenting, on a display device, one or more images of the object generated based on the determined magnetic susceptibility distribution.

As examples of specific implementations, the regularization term may involve a third and/or more regions with features of susceptibility contrasts differing from features of susceptibility contrasts of the first two regions; the feature of susceptibility contrasts of the first region may be the lowest; the regularization term may involve a penalty on a variation of susceptibility values in a region, wherein the penalty may vary with the feature of susceptibility contrasts of a region; the feature of susceptibility contrasts of a region may involve decay rates of magnitude signals in voxels of the region, such as the R2* decay rate of the measured magnetic resonance signal, or an opposite or inverse of T2* or T2 weighted image; a region may be formed by binning a group of voxels sharing a characteristic, where the characteristic is a distribution of decay rates wherein the distribution may be a rectangular or Gaussian function, and/or the characteristic may be a distribution in space; minimizing the cost function may be obtained using a preconditioning method; the regularization term may be implicitly formed and estimating the magnetic susceptibility distribution may be realized using a deep neural network, wherein the deep neural network may be trained with labeled data or unlabeled data, or is untrained, or the deep neural network may be trained with network weights updated with a test data; modeling a magnetic susceptibility effect on the obtained complex magnetic resonance data may involve a comparison between the obtained complex magnetic resonance data and a susceptibility modeled complex signal at each echo time, wherein the susceptibility modeled complex signal may include a phase involving echo time and the magnetic susceptibility dipole convolution, and/or the comparison comprises an Lp norm (p≥0, for example, p=0 for Gaussian noise in complex data) of the difference between the obtained complex magnetic resonance data and a susceptibility modeled complex signal at each echo time; modeling a magnetic susceptibility effect on the obtained complex magnetic resonance data may comprise calculating a magnetic field experienced by tissue in the object from the obtained magnetic resonance data, calculating a phase at each echo time according to the calculated magnetic field and a multiplicative scaling factor, performing a comparison involving the calculated phase with a phase of the magnetic susceptibility dipole convolution at each echo time, and dividing the determined magnetic susceptibility distribution by the scaling factor, wherein the comparison involving the calculated phase with a phase of the magnetic susceptibility dipole convolution at each echo time may comprise calculating an Lp norm of a weighted difference between an exponential factor of the calculated phase and an exponential factor of the phase of the magnetic susceptibility dipole convolution at each echo time.

In an example implementation, the data fidelity term is associated with a comparison between the obtained complex magnetic resonance imaging data and a susceptibility modeled complex signal at each echo time wherein the susceptibility modeled complex signal has echo time dependence in both signal magnitude and phase with the signal phase echo time dependence including the magnetic susceptibility dipole convolution.

The magnetic susceptibility distribution may be determined by minimizing the cost function. The minimization procedure may involve the use of preconditioning for uniform convergence over the whole imaging volume. The determination of the susceptibility distribution may be performed using a deep neural network. The data fidelity term may involve a comparison between the measured and a susceptibility modeled complex signal at each echo time wherein the susceptibility modeled complex signal has echo time dependence in both signal magnitude and phase. An example for the complex signal model is that signal magnitude decays with echo time exponentially with a decay rate of R2*, and signal phase evolves linearly with echo time according to the dipole convolution generated magnetic field. An example for the comparison is a difference expressed in an Lp norm. The magnetic susceptibility distribution may also be determined by calculating a magnetic field experienced by the tissue in the object based on modeling the multiecho complex data with consideration of noise in both magnitude and phase information at each echo time, replacing the temporal phase evolution with echo time in the measured multiecho complex data with a phase factor determined by the calculated magnetic field; and dividing the calculated magnetic field by a factor for estimating the magnetic susceptibility and then the final output magnetic susceptibility is multiplied by the factor.

In general, another implementation for generating one or more images of an object comprises obtaining complex magnetic resonance imaging data collected by a magnetic resonance scanner wherein the data is complex and constitutes magnitude and phase information regarding the object, estimating a magnetic susceptibility distribution of the object based on the complex data by determining a cost function that includes a data fidelity term involving the magnetic susceptibility dipole convolution modeling of the data phase and a regularization term involving penalties on susceptibility variation in at least two regions of low susceptibility contrasts and by determining a magnetic susceptibility distribution based on minimizing the cost function.

In general, another implementation for generating one or more images of an object comprises obtaining multiecho complex magnetic resonance imaging data collected by a magnetic resonance scanner, wherein the multiecho data is complex and constitutes magnitude and phase information regarding the object, and estimating fat and water distribution of the object based on the multiecho complex data by determining a cost function to include a data fidelity term involving a comparison between the measured and a complex signal of a fat-water model at each echo time wherein the fat-water model has echo time dependence in both signal magnitude and phase with the fat chemical shift contributing an additional phase. The cost function is minimized using a deep neural network without training of labeled data.

As examples of specific implementations, the comparison between the obtained multiecho complex magnetic resonance data and a complex signal of a fat-water model at each echo time comprises an Lp norm of a difference between the obtained multiecho complex magnetic resonance data and a complex signal of a fat-water model at each echo time; the fat-water model may have echo time dependence in signal magnitude with different decay rates for fat and water components; the fat chemical shift contributing an additional phase may comprise a sum of contributions from a fat chemical shift spectrum; the deep neural network may be trained with unlabeled data, wherein network weights of the trained deep neural network may be updated with a test data, and/or the deep neural network may be untrained; the deep neural network may include a U-net.

In general, another implementation for generating one or more images of an object comprises obtaining multiecho complex magnetic resonance imaging data collected by a magnetic resonance scanner, wherein the multiecho data is complex and constitutes magnitude and phase information regarding the object, and a magnetic susceptibility distribution is calculated using quantitative susceptibility mapping processing of the obtained multiecho complex magnetic resonance imaging data, and estimating oxygen extraction fraction distribution of the object based on the multiecho complex data by determining a cost function to include a data fidelity term involving a comparison between a magnitude image of obtained multiecho complex magnetic resonance imaging data and a magnitude image of a modeled complex signal with a consideration of deoxyheme-iron effects at each echo time, and a comparison between the calculated susceptibility and a susceptibility decomposition model including a deoxyheme-iron component, and minimizing the cost function using a deep neural network.

In general, another implementation for generating one or more images of an object comprises obtaining multiecho complex magnetic resonance imaging data collected by a magnetic resonance scanner, wherein the multiecho data is complex and comprises magnitude and phase information regarding the object, calculating a magnetic susceptibility distribution using quantitative susceptibility mapping processing of the obtained multiecho complex magnetic resonance imaging data, determining oxygen extraction fraction distribution of the object based on the multiecho complex data, comprising determining a cost function, the cost function involving a comparison between a magnitude image of obtained multiecho complex magnetic resonance imaging data and a magnitude image of a modeled complex signal with a consideration of deoxyheme-iron effects at each echo time, and a comparison between the calculated susceptibility and a susceptibility decomposition model including a deoxyheme-iron component, and a regularization term on oxygen extraction fraction and venous blood volume, and minimizing the cost function, and presenting, on a display device, one or more images of the object generated based on the determined oxygen extraction fraction distribution.

In general, another implementation for generating one or more images of an object comprises obtaining multiecho complex magnetic resonance imaging data collected by a magnetic resonance scanner, wherein the multiecho data is complex and comprises magnitude and phase information regarding the object, determining oxygen extraction fraction distribution of the object based on the multiecho complex data, comprising determining a cost function, the cost function including a data fidelity term involving a comparison between the obtained multiecho complex magnetic resonance imaging data and a modeled complex signal at each echo time with a consideration of deoxyheme-iron effects on signal magnitude and phase, and a regularization term, and minimizing the cost function, and presenting, on a display device, one or more images of the object generated based on the determined oxygen extraction fraction distribution.

As examples of specific implementations, the consideration of deoxyheme-iron effects on signal magnitude and phase may include a model of deoxyheme-iron distributed in cylinders and nonblood susceptibility sources distributed diffusely in space for determining the modeled complex signal; the comparison between the obtained multiecho complex magnetic resonance data and the modeled complex signal may comprise an Lp norm of the difference of the obtained multiecho complex magnetic resonance data and the modeled complex signal. The one or more images of the object include the nonblood susceptibility distribution and/or venous blood volume; the regularization term may include a sparsity of edges in oxygenation and/or venous blood volume; minimizing the cost function may include a dipole deconvolution step and the regularization term includes a sparsity of edges in tissue susceptibility and/or penalties on variations of susceptibility values in regions; the regularization term may include a sparsity of temporal evolution along echo time of the magnitude of the modeled complex signal; minimizing the cost function may include coordinate descent along axes defined by variables of oxygenation and venous blood volume; the regularization term may be implicitly formed and determining the oxygen extraction fraction distribution is realized using a deep neural network, wherein the deep neural network may be trained with labeled data or unlabeled data, or is untrained, and/or the deep neural network is trained with network weights updated with a test data.

The modeled complex signal with a consideration of deoxyheme-iron effects on signal magnitude and phase at each echo time include modeling deoxyheme-iron as cylinders and other susceptibility sources as diffuse and calculating corresponding effects on signal magnitude and phase at each echo time. Deep neural networks can be used to minimize the cost function.

In general, another implementation for generating one or more images of an object comprises obtaining multiple timeframe imaging data about tracer transport through the object, determining tracer concentration at each timeframe from imaging data according to tracer effects on image signal, estimating tracer velocity and exchange coefficient distribution of the object by determining a cost function that includes a data fidelity term involving a comparison between the determined tracer concentrations and tracer concentrations according to a velocity-exchange model, minimizing the cost function. determining a velocity and exchange distribution based on the act of minimizing the cost function, and presenting, on a display device, one or more images of the object generated based on the determined velocity and/or exchange distribution.

As examples of specific implementations, the multiple timeframe imaging data may be obtained using magnetic resonance imaging, positron emission tomography, or computed tomography, or single photon emission tomography; the velocity-exchange model may include a temporal deconvolution involving an exchange coefficient; the comparison between the obtained tracer concentrations and tracer concentrations according to a velocity-exchange model may comprise an Lp norm of a difference between the obtained tracer concentrations and tracer concentrations according to a velocity-exchange model; the cost function may include a regularization term imposing a sparsity on velocity and/or exchange coefficient distribution; the regularization term may be implicitly formed and determining the velocity and exchange coefficient distribution is realized using a deep neural network, wherein the deep neural network may be trained with labeled data or unlabeled data, or is untrained, or the deep neural network is trained with network weights updated with a test data.

The velocity-exchange model for tracer passage through tissue is formulated in a transport equation accounting for tracer convection with a velocity and tracer binding with tissue. The cost function includes a regularization on the velocity and exchange coefficient, with the regularization formulated explicitly such as using sparsity and/or implicitly using a deep neural network. The cost function can be minimized using gradient descent or using a deep neural network.

For each general or specific implementations described in the above, there is a system for generating one or more images of an object, the system comprising a processor, a graphical output module communicatively coupled to the processor, an input module communicatively coupled to the processor, and a non-transitory computer storage medium encoded with a computer program, the program comprising instructions that when executed by processor cause the processor to perform operations to realize the implementation.

1. Multiecho Complex Total Field Inversion (mcTFI) Method for Improved Signal Modeling in QSM

In this section, we present an improvement on constructing QSM by modeling the MRI signal more precisely, particularly accounting for noise in signal amplitude that has been ignored in the prior art.

### 1.1. Introduction

Quantitative Susceptibility Mapping (QSM) is an MRI contrast mechanism that is capable of the mapping of the underlying tissue magnetic susceptibility. The magnetic susceptibility sources are magnetized when placed inside an external magnetic field such as the main field B0 of an MRI scanner, and the magnetic fields generated by the magnetized susceptibility sources, henceforth termed the susceptibility field, can then be measured by the MRI scanner for susceptibility map reconstruction. Accordingly, QSM can be used to study tissue susceptibility sources such as deoxyheme iron in blood, tissue nonheme iron, myelin, cartilage, and calcification. QSM robustness and reproducibility have been demonstrated for brain applications, and there are a wide range of QSM clinical applications, including deep brain stimulation target mapping, cerebral cavernous malformation monitoring, multiple sclerosis chronic inflammation imaging and MRI follow-up without gadolinium, neurodegeneration investigation in Parkinson's disease and Alzheimer's disease, and more.

Technical challenges remain for QSM in regions of low SNR, such as lesions with large susceptibility contrasts and regions near air-tissue interfaces, and these challenges become more problematic at higher field strengths. Typically, the multiecho gradient-echo (GRE) sequence is used for QSM data acquisition and the susceptibility field can be inferred as the change in the phase of the GRE images measured at the multiple echo times. QSM reconstruction comprises first estimating this susceptibility field from the GRE images, and then reconstructing the susceptibility map from the estimated field. While proper noise modeling of the estimated field is important, assumptions made during field estimation from GRE data may introduce errors. These errors can propagate into the reconstructed susceptibility map as artifacts. The MERIT method used for iterative tuning of the noise weighting helps to alleviate some of the resulting artifacts, but its implementation remains empirical.

Here we report a multiecho Complex Total Field Inversion (mcTFI) method to compute the susceptibility map directly from the acquired GRE images using an improved signal model and the Gaussian noise property of the complex data. We compared mcTFI with preconditioned Total Field Inversion (TFI), to demonstrate improvements in susceptibility reconstruction, especially in regions with low SNR.

### 1.2 Theory

In this work, the gradient echo signal Si measured at echo time tⱼ with j=1, . . . , #TE is modeled as:

Sⱼ=m₀e^{-tjR*}^{₂}e^{iϕ}^{₀}e^{itj2πf} [3]

Where m₀ is the initial magnetization, R*₂ is the T₂ relaxation rate, ϕ₀ is the initial phase offset, and f is the susceptibility field (in a left-handed phase convention, which is equivalent to a right-handed phase convention but with opposite sign). The susceptibility field is modeled as a dipole field: it is the convolution of a susceptibility distribution x with the magnetic field of a unit dipole. An operator D=d*is used to denote the convolution with the unit dipole field:

f= [symbol] B₀d*x [4]

Where ω₀=γB₀ with B₀ the main magnetic field. Therefore, Eq. 3 can also be written as an example for modeling a magnetic susceptibility effect on complex magnetic resonance data. The susceptibility modeled complex signal at the j^{th} echo is:

Sⱼ=m₀e^{-t}^{ⱼ}^{R*}^{₂}e^{iϕ}^{₀}e^{it}^{ⱼ}^{ω}^{₀}^{Dχ} [5]

The goal of QSM is to reconstruct the susceptibility distribution map, χ, from the measured complex signal (GRE images) at the j^{th} echo, Sⱼ.

Here, we provide mcTFI to compute the susceptibility distribution directly from the complex gradient echo data. As an exemplary embodiment for the cost function relating at least a data fidelity term comparing the measured complex signal and the susceptibility modeled signal. For example, the data fidelity term can be associated with a Gaussian likelihood function involving a difference between the measured and a susceptibility modeled complex signal at each echo time wherein the susceptibility modeled complex signal has time dependence in both signal magnitude and phase, and a regularization term associated with a structure prior information, we formulate the following cost function:

$$[ 6 ]$$

$${χ}^{′} = {Py}^{′} , ( {m}_{0}^{′} , {R}_{2}^{{*}^{′}} , {ϕ}_{0}^{′} , {Py}^{′} ) = {argmin}_{{m}_{0} , {R}_{2}^{*} , {ϕ}_{0} , y} {\sum }_{j = 1}^{\# TE} ‖ {m}_{0} e {- {t}_{j} {R}_{2}^{*}}^{} {e}^{i {ϕ}_{0}} {e}^{{it}_{j} DPy} {- {S}_{j} ‖}_{2}^{2} + \\ {λ}_{1} {‖ {M}_{G} ∇ Py ‖}_{1} + {λ}_{2} {‖ {M}_{c} ( Py - {\overline{Py}}^{{M}_{c}} ) ‖}_{2}^{2} + {λ}_{3} R ( {m}_{0} , {R}_{2}^{*} , {ϕ}_{0} )$$

Where m₀, R*₂, and ϕ₀, were solved alongside with the susceptibility map, χ=Py, with P being a preconditioner. The second term is a weighted total variation designed to suppress streaking artifacts in y, where λ₁ is the regularization parameter, M_{G} is the binary edge mask reflecting the anatomy, and ∇ is the gradient operator. The third term, also known as the zero-reference term, enforces constant susceptibility distribution in Py within a mask M_{c} where λ₂ is the regularization parameter, and P̅y̅^{Mc}, is the average of Py over M_{c}. In brain QSM, M_{c} is typically chosen as the regions containing CSF. The last term R(m₀, R*₂, ϕ₀) is a regularization, such as a Tikhonov regularization on the updates of m₀, R*₂, and ϕ₀ to improve stability

$$R = {‖ \begin{matrix}d {m}_{0} \\ {dR}_{2}^{*} \\ d {ϕ}_{0}\end{matrix} ‖}_{2}^{2} ,$$

where λ₃ is the regularization parameter.

The optimization problem in Eq. 6 was solved iteratively using the Gauss-Newton (GN) method. In each GN iteration, Eq. 6 was linearized with respect to the four unknowns via first order Taylor expansion. For computational efficiency, the linearized problem at the nth GN iteration was broken down into two subproblems using coordinate descending approach: 1) keeping m_{0,n}, R*_{2,n}, and ϕ_{0,n} fixed, yₙ₊₁ was found using the Conjugate Gradient (CG) solver (see Appendix A in the next section 1.3 for solver detail):

$${y}_{n + 1}^{′} = {argmin}_{y} {\sum }_{j = 1}^{\# TE} {‖ {m}_{0 , n} {e}^{- {t}_{j} {R}_{2 , n}^{*}} {e}^{i {ϕ}_{0 , n}} {e}^{{it}_{j} DPy} - {S}_{j} ‖}_{2}^{2} + {λ}_{1} {‖ {M}_{G} ∇ Py ‖}_{1} + {λ}_{2} {‖ {M}_{c} ( Py - {\overline{Py}}^{{M}_{c}} ) ‖}_{2}^{2} \qquad [ 7 ]$$

and 2) keeping yₙ₊₁ fixed, m_{0,n+1}, R*_{2,n+1}, and ϕ_{0,n+1} were updated using a voxel-by-voxel pseudo-inversion (see Appendix B in the next section 1.3 for solver detail):

$$( {m}_{0 , n + 1}^{′} , {R}_{2 , n + 1}^{* ′} , {ϕ}_{0 , n + 1}^{′} ) = {argmin}_{{m}_{0} , {R}_{2}^{*} , {ϕ}_{0}} {\sum }_{j = 1}^{\# TE} {‖ {m}_{0} {e}^{- {t}_{j} {R}_{2}^{*}} {e}^{i {ϕ}_{0}} {e}^{{it}_{j} {PDy}_{n + 1}} - {S}_{j} ‖}_{2}^{2} + {λ}_{3} R ( {m}_{0} , {R}_{2}^{*} , {ϕ}_{0} ) \qquad [ 8 ]$$

The mcTFI method described in the above addresses important shortcomings in the prior art of field estimation and QSM. For example, in prior TFI, QSM is computed by fitting the susceptibility to the susceptibility field, f, which is first estimated via a fitting by ignoring the noise in the signal amplitude in Eq. 7 and solving only for ϕ₀ and f:

$$( {ϕ}_{0}^{′} , {f}^{′} ) = {argmin}_{{ϕ}_{0} , f} {\sum }_{j = 1}^{\# TE} {‖ \vert {S}_{j} \vert {e}^{i {ϕ}_{0}} {e}^{{it}_{j} 2 π f} - {S}_{j} ‖}_{2}^{2} \qquad [ 9 ]$$

the susceptibility map was then reconstructed according to the following prior method that assumes Gaussian noise in the estimated field:

$${χ}^{′} = {Py}^{′} ,$$

$${y}^{′} = {argmin}_{y} {‖ w ( DPy - f ) ‖}_{2}^{2} + {λ}_{1} {‖ {M}_{G} ∇ Py ‖}_{1} + {λ}_{2} {‖ {M}_{c} ( Py - {\overline{Py}}^{{M}_{c}} ) ‖}_{2}^{2}$$

where w is a diagonal noise weighting matrix, which is typically calculated as the covariance matrix of the fitting in Eq. 9 with the assumption of Gaussian phase at each echo in a multiecho gradient echo sequence. Note that the noise in signal magnitude may be substantial, and the noise in f may no longer be Gaussian, which could introduce model errors to the data fidelity term in the above prior method. Using a proper noise weighting in TFI can help to mitigate this model error (as well as fitting errors from Eq. 9) by assigning small values to w in the problematic voxels, therefore, reducing the influence of these problematic voxels during the optimization. To address that w can also contain errors and may not completely account for the noise property in the susceptibility field, MERIT is introduced to further retune w. With MERIT, at the end of the ith GN iteration, the noise weighting wᵢ is updated to wᵢ₊₁, which will be used in the next GN iteration, as follows:

$${w}_{i + 1} = {w}_{i} if \frac{{ρ}_{i}}{T} \leq 1 , {{w}_{i} ( \frac{{ρ}_{i}}{T} )}^{- R}$$

otherwise where ρᵢ=wᵢ|DPyᵢ−f| is the voxel-by-voxel data term residual for the iₜₕ iteration, T is the threshold determining the number of voxels whose noise weighting will be reduced, and R is the attenuation factor determining the strength of the weighting reduction. Carefully selection of T and R are important: large values may cause reasonable voxels to be disregarded and small values may not probably penalize all erroneous voxels. However, MERIT is a heuristic method and the parameters, T and R, are currently empirically determined.

The proposed mcTFI method is hypothesized to be an improvement upon the TFI method because 1) mcTFI does not require the fine-tuning of w, and 2) by avoiding a separate fitting of f, the Gaussian noise assumption in mcTFI cost function still holds.

### 1.3 Materials and Methods

The accuracy of the proposed mcTFI algorithm was evaluated against TFI both without and with MERIT (TFI+MERIT) in a numerical brain simulation with inclusion of a hemorrhage and calcification, in patients suffering from intracerebral hemorrhage (ICH) scanned at 3T and in healthy subjects scanned at 7T.

### Implementation Details

In TFI, P is the automated adaptive preconditioner, λ₁=0.001, and λ₂=0.1 were chosen as accordance with the literature. The MERIT parameters T=6σ, where σ is the standard deviation of the two-norm of the data term residual, and R=2 were chosen according to the original work.

The same automated adaptive preconditioner was used in mcTFI. The follows steps were taken in order to match the regularizations parameters in mcTFI to that in TFI:
1) The noise weighting in TFI was scaled such that its mean values over the brain mask M was 1, or

$$\frac{1}{\# M} {\sum }_{k ∈ M} {w}_{k} = 1$$

with #M the number of voxels in M. In mcTFI, the complex images, Sⱼ were scaled by

$$C = \frac{1}{\# TE} {\sum }_{i = 1}^{\# TE} \frac{1}{\# M} {\sum }_{k ∈ M} \vert {S}_{j , k} \vert ,$$

so that

$$\frac{1}{\# TE} {\sum }_{j = 1}^{\# TE} \frac{1}{\# M} {\sum }_{k ∈ M} \vert \frac{{S}_{j , k}}{C} \vert = 1 .$$

2) λ_{1,mcTFI}=λ_{1,TFI}×Σⱼ₌₁^{#TE}tⱼ and λ_{2,mcTFI}=λ_{2,TFI}×Σⱼ₌₁^{#TE}tⱼ² were chosen as the regularization parameters (see appendix A in this section 1.3 below for derivation).

The Tikhonov regularization parameter, λ₃, was automatically determined for each case by first constructing a L-curve in a large range, from 10⁻⁵ to 10¹ with an increment of an order of magnitude, to determine a rough value for the L-curve corner. Then, a second L-curve was constructed within a range of an order of magnitude around this rough value, and λ³ was chosen as the corner of the second L-curve. The L-curve corner was determined using an adaptive pruning algorithm. To further improve the stability of mcTFI, m₀ and R*₂ were constrained to be greater than or equal to zero.

Proper initializations are important for solving the nonlinear optimization problems in mcTFI. Here, R*_{2,init} was obtained using ARLO, and then m_{0,init} was estimated as

$${m}_{0 , init} = \frac{\vert {S}_{1} \vert}{{e}^{- {t}_{1} {R}_{2 , init}^{*}}} .$$

ϕ_{0,init} and fᵢₙᵢₜ were obtained via pseudo-inversion of the phase in the first two GRE echoes:

$$( {ϕ}_{0 , init} , {f}_{init} ) = {[ \begin{matrix}1 & {t}_{1} \\ 1 & {t}_{2}\end{matrix} ]}^{- 1} [ \begin{matrix}{∠ S}_{1} \\ {∠ S}_{2}\end{matrix} ] .$$

One challenge in initializing Eq. 6 is that the angle of a complex exponential is only defined within a range of 2π, so that large susceptibility field may cause wraps in the phase, leading in turn to wraps in the resulting susceptibility map. Such wrapping artifact can be avoided if y is initialized without any wraps, and this was done with the following steps:
1) Perform phase unwrapping on fᵢₙᵢₜ
2) Perform phase unwrapping on the phase of the GRE images:

$${y}_{nowrap} = {arg min}_{y} {\sum }_{j = 1}^{\# TE} {‖ {m}_{0 , init} {e}^{- {t}_{j} {R}_{2 , init ,}^{*}} {e}^{i {ϕ}_{0 , init ,}} {e}^{{it}_{j} DPy} - {S}_{j , nowrap} ‖}_{2}^{2} + {λ}_{1} {‖ {M}_{G} ∇ Py ‖}_{1} + {λ}_{2} {‖ {M}_{c} ( Py - {\overline{Py}}^{{M}_{c}} ) ‖}_{2}^{2} ,$$

3) Construct a new set of GRE images that do not have phase wrap: S_{j,nowrap}=|Sⱼ|e^{iα∠S}^{_{j,nowrap}}. Here, α is a very large scalar that ensures the range of α∠S_{j,unwrap} falls within 2π so that there is no phase wrap.
4) Solve

$${∠ S}_{j , nowrap} = {∠ S}_{j} - round ( \frac{{∠ S}_{j} - {f}_{init} * {t}_{j} + {ϕ}_{0 , init}}{2 π} ) * 2 π$$

linearized at y=0 and with 1 GN iteration.
5) yᵢₙᵢₜ=y_{nowrap}/α

A quality map guided spatial unwrapping algorithm was used for phase unwrapping.

### Simulation

A numerical brain phantom with calcification and hemorrhage was constructed based on the Zubal phantom. First, multiecho GRE image data (ignoring T1 relaxation effect) were simulated according to Eq. 5, on 3T, with simulation parameters 1^{st} TE (ms)/ΔTE (ms)/#TE (ms)/voxel size (mm³)=4.5/5/8/1×1×1, and with known tissue parameters m₀ (a.u.)/R*₂(Hz)/ϕ₀ (rad)/χ (ppm) set for different structures based on literature values: white matter=0.715/20/1/−0.046, grey matter=0.715/20/1/0.053, CSF=0.715/4/1/0, caudate nucleus=0.715/30/1/0.093, putamen=0.715/30/1/0.093, thalamus=0.9/20/1/0.073, globus pallidus=0.9/45/1/0.193, calcification=0.1/500/1/−2.4, ICH=0.1/150/1/2.68, air=0/0/0/9, sagittal sinus=0/45/0/0.27, and skull=0/4/0/−2. Next, random zero-mean gaussian white noise at SNR=100 was added to the real and imaginary parts of the complex image independently. Finally, the GRE images were downsampled by a factor of 2, from which the QSM images were reconstructed using the different methods for comparison. This simulation experiment was repeated for SNR ranging from 10 to 150 to evaluate the performance of the QSM methods at different SNR levels, and at each SNR level the experiment was repeated 10 times. The accuracy of the reconstructed QSM was calculated as the mean of the

$$RMSE = \sqrt{\frac{1}{N} {\sum }_{i}^{N} {( {M}_{{χ}_{truth , i}} - {M}_{{χ}_{recon , i}} )}^{2}}$$

over the 10 repeats; RMSE was calculated inside of the brain mask, only inside of the lesions, and only outside of the lesions. The lesion mask was set as the actual lesion plus 3 layers of voxels around it.

### QSM Challenge 2.0 Data

The 4 datasets (1 brain with and without calcification, simulated at two different SNR) provided in the QSM Challenge 2.0 were used to compare mcTFI, TFI, and TFI+MERIT. The results were evaluated according to the metrics of QSM Challenge 2.0, which are:
1) RMSE over the whole brain
2) demeaned and detrended RMSE, ddRMSE, calculated over the whole brain, over the cortical gray and white matter, over the deep gray matter, and over the venous blood
3) linear regression analysis over all voxels in deep gray matter
4) streaking artifact level around calcification
5) the magnetic moment of the calcification

Both data and evaluation codes are available on the official QSM Challenge 2.0 website.

In vivo Brain

Eighteen ICH patients were scanned on a commercial 3T scanner (750/SIGNA, General Electric Healthcare, Waukesha Wisconsin, USA) with a unipolar flow compensated multiecho gradient echo sequence. The acquisition parameters were: FA=15, FOV=25.6 cm, TE1=4.5-5.0 ms, TR=39-49 ms, #TE=6-8, ΔTE=4.6-5 ms, acquisition matrix=512×512×64-144, reconstructed voxel size=0.5×0.5 mm², reconstructed slice thickness=1-3 mm, and BW=±62.5 kHz.

Six healthy volunteers were scanned on a prototype 7T MR scanner (MR950, Signa 7.0T, General Electric Healthcare, Waukesha, WI) using a two-channel transmit/32-channel to receive head coil with a multiecho gradient echo sequence. The acquisition parameters were: FA=15, FOV=220×176 mm², TE1=3.81 ms, TR=45.03 ms, #TE=10, ΔTE=4.1 ms, acquisition matrix=320×320×74, reconstructed voxel size=0.5×0.5 mm², reconstructed slice thickness=1 mm, and BW=±78.1 kHz.

The image quality of the reconstructed hemorrhage was scored semi-quantitatively based on visually assessed artifacts with a three-point scale (3=severe, 2=moderate, and 1=negligible), by an experienced radiologist. The image quality scores (presented as mean±standard deviation) were compared with each other using a two-tailed Wilcoxon paired-sample signed rank test, and a two-sided p<0.05 was deemed indicative of statistical significance.

Appendix A

Starting from Eq. 7, let Wⱼ=m₀e^{-t}^{ⱼ}^{R*}^{₂}e^{iϕ}^{₀}e^{it}^{ⱼ}^{DPy}, and APy=Py−P̅y̅^{M}^{_{c}}:

$$E ( y ) = {arg min}_{y} {\sum }_{j = 1}^{\# TE} {‖ {W}_{j} - {S}_{j} ‖}_{2}^{2} + {λ}_{1} {‖ {M}_{G} ∇ Py ‖}_{1} + {λ}_{2} {‖ {M}_{c} APy ‖}_{2}^{2}$$

First order Taylor expansion around y gives:

$$E ( y + dy ) = {\sum }_{j = 1}^{\# TE} {‖ \begin{matrix}{W}_{j} - {S}_{j} \\ + {W}_{j} ( {it}_{j} DPdy )\end{matrix} ‖}_{2}^{2} + {λ}_{1} {‖ {M}_{G} ∇ P ( y + dy ) ‖}_{1} + {λ}_{2} {‖ {M}_{c} AP ( y + dy ) ‖}_{2}^{2}$$

The regularizations terms can be reformatted as follows so that the regularizations terms at each j can be scaled down by tⱼ:

$$E ( y + dy ) = {\sum }_{j = 1}^{\# TE} {‖ \begin{matrix}{W}_{j} - {S}_{j} \\ + {W}_{j} ( {it}_{j} DPdy )\end{matrix} ‖}_{2}^{2} + {λ}_{1} {\sum }_{j = 1}^{\# TE} {‖ {t}_{j} {M}_{G} ∇ P ( y + dy ) ‖}_{1} + {λ}_{2} {\sum }_{j = 1}^{\# TE} {‖ {t}_{j} {M}_{c} AP ( y + dy ) ‖}_{2}^{2}$$

$$Let {B}_{j} = {W}_{j} - {S}_{j} :$$

$$E ( y + dy ) = {\sum }_{j = 1}^{\# TE} {‖ {B}_{j} + {W}_{j} ( {it}_{j} DPdy ) ‖}_{2}^{2} + {λ}_{1} {\sum }_{j = 1}^{\# TE} {‖ {t}_{j} {M}_{G} ∇ P ( y + dy ) ‖}_{1} + {λ}_{2} {\sum }_{j = 1}^{\# TE} {‖ {t}_{j} {M}_{c} AP ( y + dy ) ‖}_{2}^{2}$$

Gradient of E with respect to dy:

$$\frac{δ E}{δ dy} = \frac{δ}{δ dy} ( \begin{matrix}{\sum }_{j = 1}^{\# TE} {dy}^{H} {P}^{H} {D}^{H} {t}_{j}^{H} {i}^{H} {W}_{j}^{H} {W}_{j} {it}_{j} DPdy + {dy}^{H} {P}^{H} {D}^{H} {t}_{j}^{H} {i}^{H} {W}_{j}^{H} {B}_{j} + {B}_{j}^{H} {W}_{j} {it}_{j} DPdy \\ {λ}_{1} {\sum }_{j = 1}^{\# TE} \vert {t}_{j} {M}_{G} ∇ P ( y + dy ) \vert \\ {λ}_{2} {\sum }_{j = 1}^{\# TE} {dy}^{H} {P}^{H} {A}^{H} {M}^{H} {t}_{j}^{H} {t}_{j} MAPdy + {dy}^{H} {P}^{H} {A}^{H} {M}^{H} {t}_{j}^{H} {t}_{j} MAPy + {y}^{H} {P}^{H} {A}^{H} {M}^{H} {t}_{j}^{H} {t}_{j} MAPdy\end{matrix} ) = ( \begin{matrix}{\sum }_{j = 1}^{\# TE} 2 Re \{ {P}^{H} {D}^{H} {t}_{j}^{H} {i}^{H} {W}_{j}^{H} {W}_{j} {it}_{j} DPdy \} + 2 Re \{ {P}^{H} {D}^{H} {t}_{j}^{H} {i}^{H} {W}_{j}^{H} {B}_{j} \} \\ {λ}_{1} {\sum }_{j = 1}^{\# TE} {P}^{H} {∇}^{H} {M}_{G}^{H} {t}_{j}^{H} \frac{1}{\vert {t}_{j} {M}_{G} ∇ Py \vert} [ {t}_{j} {M}_{G} ∇ P ( y + dy ) ] \\ {λ}_{2} {\sum }_{j = 1}^{\# TE} 2 {P}^{H} {A}^{H} {M}^{H} {t}_{j}^{H} {t}_{j} MAPdy + 2 {P}^{H} {A}^{H} {M}^{H} {t}_{j}^{H} {t}_{j} MAPy\end{matrix} )$$

In both λ₁ and λ₂ terms, since tⱼ is the only summation variable and tⱼ is scalar, the above expression can be further simplified:

$$\frac{δ E}{δ dy} = ( \begin{matrix}{\sum }_{j = 1}^{\# TE} 2 Re \{ {P}^{H} {D}^{H} {t}_{j}^{H} {i}^{H} {W}_{j}^{H} {W}_{j} {it}_{j} DPdy \} + 2 Re \{ {P}^{H} {D}^{H} {t}_{j}^{H} {i}^{H} {W}_{j}^{H} {B}_{j} \} \\ {λ}_{1} ( {\sum }_{j = 1}^{\# TE} {t}_{j} ) {P}^{H} {∇}^{H} {M}_{G}^{H} \frac{1}{\vert {M}_{G} ∇ Py \vert} [ {M}_{G} ∇ P ( y + dy ) ] \\ {λ}_{2} ( {\sum }_{j = 1}^{\# TE} {t}_{j}^{2} ) 2 {P}^{H} {A}^{H} {M}^{H} MAPdy + 2 {P}^{H} {A}^{H} {M}^{H} MAPy\end{matrix} )$$

Settings the gradients to zero:

$$( \begin{matrix}{\sum }_{j = 1}^{\# TE} 2 Re \{ {P}^{H} {D}^{H} {t}_{j}^{H} {i}^{H} {W}_{j}^{H} {W}_{j} {it}_{j} DPdy \} \\ {λ}_{1} ( {\sum }_{j = 1}^{\# TE} {t}_{j} ) {P}^{H} {∇}^{H} {M}_{G}^{H} \frac{1}{\vert {M}_{G} ∇ Py \vert} [ {M}_{G} ∇ Pdy ] \\ {λ}_{2} ( {\sum }_{j = 1}^{\# TE} {t}_{j}^{2} ) 2 {P}^{H} {A}^{H} {M}^{H} MAPdy\end{matrix} ) = ( \begin{matrix}{\sum }_{j = 1}^{\# TE} 2 Re \{ {P}^{H} {D}^{H} {t}_{j}^{H} {i}^{H} {W}_{j}^{H} {B}_{j} \} \\ {λ}_{1} ( {\sum }_{j = 1}^{\# TE} {t}_{j} ) {P}^{H} {∇}^{H} {M}_{G}^{H} \frac{1}{\vert {M}_{G} ∇ Py \vert} [ {M}_{G} ∇ Py ] \\ {λ}_{2} ( {\sum }_{j = 1}^{\# TE} {t}_{j}^{2} ) 2 {P}^{H} {A}^{H} {M}^{H} MAPy\end{matrix} )$$

Which can be written as

$$( \begin{matrix}2 Re \{ {P}^{H} {D}^{H} {W}_{1} DPdy \} \\ {λ}_{1} ( {\sum }_{j = 1}^{\# TE} {t}_{j} ) {P}^{H} {∇}^{H} {M}_{G}^{H} \frac{1}{\vert {M}_{G} ∇ Py \vert} [ {M}_{G} ∇ Pdy ] \\ {λ}_{2} ( {\sum }_{j = 1}^{\# TE} {t}_{j}^{2} ) 2 {P}^{H} {A}^{H} {M}^{H} MAPdy\end{matrix} ) = ( \begin{matrix}2 Re \{ {P}^{H} {D}^{H} {W}_{2} \} \\ {λ}_{1} ( {\sum }_{j = 1}^{\# TE} {t}_{j} ) {P}^{H} {∇}^{H} {M}_{G}^{H} \frac{1}{\vert {M}_{G} ∇ Py \vert} [ {M}_{G} ∇ Py ] \\ {λ}_{2} ( {\sum }_{j = 1}^{\# TE} {t}_{j}^{2} ) 2 {P}^{H} {A}^{H} {M}^{H} MAPy\end{matrix} )$$

$$With$$

$${W}_{1} = {\sum }_{j = 1}^{\# TE} {t}_{j}^{H} {i}^{H} {W}_{j}^{H} {W}_{j} {it}_{j} = {\sum }_{j = 1}^{\# TE} {t}_{j}^{2} {\vert {W}_{j} \vert}^{2}$$

$$and$$

$${W}_{2} = {\sum }_{j = 1}^{\# TE} {t}_{j}^{H} {i}^{H} {W}_{j}^{H} {B}_{j} = {\sum }_{j = 1}^{\# TE} {t}_{j} {W}_{j}^{H} {B}_{j} .$$

Appendix B

$$( {m}_{0}^{′} , {R}_{2}^{{*}^{′}} , {ϕ}_{0}^{′} ) = {arg min}_{{m}_{0} , {R}_{2}^{*} , {ϕ}_{0}} {\sum }_{j = 1}^{\# TE} {‖ {m}_{0} {e}^{- {t}_{j} {R}_{2}^{*}} {e}^{i {ϕ}_{0}} {e}^{{it}_{j} {PDy}_{n + 1}} - {S}_{j} ‖}_{2}^{2} + {λ}_{3} {‖ \begin{matrix}{dm}_{0} \\ {dR}_{2}^{*} \\ d {ϕ}_{0}\end{matrix} ‖}_{2}^{2}$$

First order Taylor expansion:

$$E ( {m}_{0} + {dm}_{0} , {R}_{2}^{*} + {dR}_{2}^{*} , {ϕ}_{0} + d {ϕ}_{0} ) = {\sum }_{j = 1}^{\# TE} {‖ \begin{matrix}{m}_{0} {e}^{- {t}_{j} {R}_{2}^{*}} {e}^{i {ϕ}_{0}} {e}^{{it}_{j} {DPy}_{n + 1}} - {S}_{j} \\ + {m}_{0} {e}^{- {t}_{j} {R}_{2}^{*}} {e}^{i {ϕ}_{0}} {e}^{{it}_{j} {DPy}_{n + 1}} ( \frac{{dm}_{0}}{{m}_{0}} ) \\ + {m}_{0} {e}^{- {t}_{j} {R}_{2}^{*}} {e}^{i {ϕ}_{0}} {e}^{{it}_{j} {DPy}_{n + 1}} ( - {t}_{j} {dR}_{2}^{*} ) \\ + {m}_{0} {e}^{- {t}_{j} {R}_{2}^{*}} {e}^{i {ϕ}_{0}} {e}^{{it}_{j} {DPy}_{n + 1}} ( id {ϕ}_{0} )\end{matrix} ‖}_{2}^{2} + {λ}_{3} {‖ \begin{matrix}{dm}_{0} \\ {dR}_{2}^{*} \\ d {ϕ}_{0}\end{matrix} ‖}_{2}^{2}$$

$$Let {W}_{j} = {m}_{0} {e}^{- {t}_{j} {R}_{2}^{*}} {e}^{i {ϕ}_{0}} {e}^{{it}_{j} {DPy}_{n + 1}} , and B = {W}_{j} - {S}_{j}$$

$$E ( {m}_{0} + {dm}_{0} , {R}_{2}^{*} + {dR}_{2}^{*} , {ϕ}_{0} + d {ϕ}_{0} ) = {\sum }_{j = 1}^{\# TE} {‖ {W}_{j} ( \frac{{dm}_{0}}{{m}_{0}} - {t}_{j} {dR}_{2}^{*} + id {ϕ}_{0} ) + B ‖}_{2}^{2} + {λ}_{3} {‖ \begin{matrix}{dm}_{0} \\ {dR}_{2}^{*} \\ d {ϕ}_{0}\end{matrix} ‖}_{2}^{2} = {\sum }_{j = 1}^{\# TE} {[ {W}_{j} ( \frac{{dm}_{0}}{{m}_{0}} - {t}_{j} {dR}_{2}^{*} + id {ϕ}_{0} ) + B ]}^{H} [ {W}_{j} ( \frac{{dm}_{0}}{{m}_{0}} - {t}_{j} {dR}_{2}^{*} + id {ϕ}_{0} ) + B ] + {{λ}_{3} [ \begin{matrix}{dm}_{0} \\ {dR}_{2}^{*} \\ d {ϕ}_{0}\end{matrix} ]}^{H} [ \begin{matrix}{dm}_{0} \\ {dR}_{2}^{*} \\ d {ϕ}_{0}\end{matrix} ]$$

Gradient of E with respect to dm₀, dR*₂, and dϕ₀:

$$\frac{δ E}{δ {dm}_{0}} = \frac{δ}{δ {dm}_{0}} \text{} ( {\sum }_{j = 1}^{\# TE} [ \begin{matrix}{( \frac{{dm}_{0}}{{m}_{0}} )}^{H} {W}_{j}^{H} {W}_{j} \frac{{dm}_{0}}{{m}_{0}} - {( \frac{{dm}_{0}}{{m}_{0}} )}^{H} {W}_{j}^{H} {W}_{j} {t}_{j} {dR}_{2}^{*} + {( \frac{{dm}_{0}}{{m}_{0}} )}^{H} {W}_{j}^{H} {W}_{j} id {ϕ}_{0} + {( \frac{{dm}_{0}}{{m}_{0}} )}^{H} {W}_{j}^{H} B \\ - {( {t}_{j} {dR}_{2}^{*} )}^{H} {W}_{j}^{H} {W}_{j} \frac{{dm}_{0}}{{m}_{0}} + {( id {ϕ}_{0} )}^{H} {W}_{j}^{H} {W}_{j} \frac{{dm}_{0}}{{m}_{0}} + {( B )}^{H} {W}_{j} \frac{{dm}_{0}}{{m}_{0}}\end{matrix} ] + {λ}_{3} ( {dm}_{0}^{H} {dm}_{0} ) ) = {\sum }_{j = 1}^{\# TE} [ ( \frac{{W}_{j}^{H}}{{m}_{0}} \frac{{W}_{j}}{{m}_{0}} + {( \frac{{W}_{j}^{H}}{{m}_{0}} \frac{{W}_{j}}{{m}_{0}} )}^{H} ) {dm}_{0} - ( \frac{{W}_{j}^{H}}{{m}_{0}} {W}_{j} {t}_{j} {dR}_{2}^{*} + {( {t}_{j} {dR}_{2}^{*} )}^{H} {W}_{j}^{H} \frac{{W}_{j}}{{m}_{0}} ) + ( \frac{{W}_{j}^{H}}{{m}_{0}} {W}_{j} id {ϕ}_{0} + {( id {ϕ}_{0} )}^{H} {W}_{j}^{H} \frac{{W}_{j}}{{m}_{0}} ) + ( \frac{{W}_{j}^{H}}{{m}_{0}} B + {( B )}^{H} \frac{{W}_{j}}{{m}_{0}} ) ] + 2 {λ}_{3} {dm}_{0} = {\sum }_{j = 1}^{\# TE} [ 2 Re \{ \frac{{W}_{j}^{H}}{{m}_{0}} \frac{{W}_{j}}{{m}_{0}} {dm}_{0} \} - 2 Re \{ \frac{{W}_{j}^{H}}{{m}_{0}} {W}_{j} {t}_{j} {dR}_{2}^{*} \} + 2 Re \{ \frac{{W}_{j}^{H}}{{m}_{0}} {W}_{j} id {ϕ}_{0} \} + 2 Re \{ \frac{{W}_{j}^{H}}{{m}_{0}} B \} ] + 2 {λ}_{3} {dm}_{0}$$

$$\frac{δ E}{δ {dR}_{2}^{*}} = \frac{δ E}{δ {dR}_{2}^{*}} \text{} ( {\sum }_{j = 1}^{\# TE} [ \text{} \begin{matrix}- {dR}_{2}^{{*}^{H}} {t}_{j}^{H} {W}_{j}^{H} {W}_{j} \frac{{dm}_{0}}{{m}_{0}} + {dR}_{2}^{{*}^{H}} {t}_{j}^{H} {W}_{j}^{H} {W}_{j} {t}_{j} {dR}_{2}^{*} - {dR}_{2}^{{*}^{H}} {t}_{j}^{H} {W}_{j}^{H} {W}_{j} id {ϕ}_{0} - {dR}_{2}^{{*}^{H}} {t}_{j}^{H} {W}_{j}^{H} B \\ - {( \frac{{dm}_{0}}{{m}_{0}} )}^{H} {W}_{j}^{H} {W}_{j} {t}_{j} {dR}_{2}^{*} - {( id {ϕ}_{0} )}^{H} {W}_{j}^{H} {W}_{j} {t}_{j} {dR}_{2}^{*} - {( B )}^{H} {W}_{j} {t}_{j} {dR}_{2}^{*}\end{matrix} ] + {λ}_{3} ( {dR}_{2}^{{*}^{H}} {dR}_{2}^{*} ) ) = {\sum }_{j = 1}^{\# TE} [ - ( {t}_{j}^{H} {W}_{j}^{H} {W}_{j} \frac{{dm}_{0}}{{m}_{0}} + {( \frac{{dm}_{0}}{{m}_{0}} )}^{H} {W}_{j}^{H} {W}_{j} {t}_{j} ) + ( {t}_{j}^{H} {W}_{j}^{H} {W}_{j} {t}_{j} + {( {t}_{j}^{H} {W}_{j}^{H} {W}_{j} {t}_{j} )}^{H} ) {dR}_{2}^{*} - ( {t}_{j}^{H} {W}_{j}^{H} {W}_{j} id {ϕ}_{0} + {( id {ϕ}_{0} )}^{H} {W}_{j}^{H} {W}_{j} {t}_{j} ) - ( {t}_{j}^{H} {W}_{j}^{H} B + {( B )}^{H} {W}_{j} {t}_{j} ) ] + 2 {λ}_{3} {dR}_{2}^{*} = {\sum }_{j = 1}^{\# TE} [ - 2 Re \{ {t}_{j}^{H} {W}_{j}^{H} {W}_{j} \frac{{dm}_{0}}{{m}_{0}} \} + 2 Re \{ {t}_{j}^{H} {W}_{j}^{H} {W}_{j} {t}_{j} {dR}_{2}^{*} \} - 2 Re \{ {t}_{j}^{H} {W}_{j}^{H} {W}_{j} id {ϕ}_{0} \} - 2 Re \{ {t}_{j}^{H} {W}_{j}^{H} B \} ] + 2 {λ}_{3} {dR}_{2}^{*}$$

$$\frac{δ E}{δ d {ϕ}_{0}} = \frac{δ}{δ d {ϕ}_{0}} ( {\sum }_{j = 1}^{\# TE} [ \begin{matrix}d {ϕ}_{0}^{H} {i}^{H} {W}_{j}^{H} {W}_{j} \frac{{dm}_{0}}{{m}_{0}} - d {ϕ}_{0}^{H} {i}^{H} {W}_{j}^{H} {W}_{j} {t}_{j} {dR}_{2}^{*} + d {ϕ}_{0}^{H} {i}^{H} {W}_{j}^{H} {W}_{j} id {ϕ}_{0} + d {ϕ}_{0}^{H} {i}^{H} {W}_{j}^{H} B \\ + {( \frac{{dm}_{0}}{{m}_{0}} )}^{H} {W}_{j}^{H} {W}_{j} id {ϕ}_{0} - {( {t}_{j} {dR}_{2}^{*} )}^{H} {W}_{j}^{H} {W}_{j} id {ϕ}_{0} + {( B )}^{H} {W}_{j} {t}_{j} id {ϕ}_{0}\end{matrix} ] + {λ}_{3} ( d {ϕ}_{0}^{H} d {ϕ}_{0} ) ) = {\sum }_{j = 1}^{\# TE} [ ( {i}^{H} {W}_{j}^{H} {W}_{j} \frac{{dm}_{0}}{{m}_{0}} + {( \frac{{dm}_{0}}{{m}_{0}} )}^{H} {W}_{j}^{H} {W}_{j} i ) - ( {i}^{H} {W}_{j}^{H} {W}_{j} {t}_{j} {dR}_{2}^{*} + {( {t}_{j} {dR}_{2}^{*} )}^{H} {W}_{j}^{H} {W}_{j} i ) + ( {i}^{H} {W}_{j}^{H} {W}_{j} i + {( {i}^{H} {W}_{j}^{H} {W}_{j} i )}^{H} ) d {ϕ}_{0} + ( {i}^{H} {W}_{j}^{H} B + {( B )}^{H} {W}_{j} i ) ] + 2 {λ}_{3} d {ϕ}_{0} = {\sum }_{j = 1}^{\# TE} [ 2 Re \{ {i}^{H} {W}_{j}^{H} {W}_{j} \frac{{dm}_{0}}{{m}_{0}} \} - 2 Re \{ {i}^{H} {W}_{j}^{H} {W}_{j} {t}_{j} {dR}_{2}^{*} \} + 2 Re \{ {i}^{H} {W}_{j}^{H} {W}_{j} id {ϕ}_{0} \} + 2 Re \{ {i}^{H} {W}_{j}^{H} B \} ] + 2 {λ}_{3} d {ϕ}_{0}$$

Settings the gradients to zero:

$${\sum }_{j = 1}^{\# TE} [ R e \{ \frac{{W}_{j}^{H}}{{m}_{0}} {W}_{j} ( \frac{d {m}_{0}}{{m}_{0}} - {t}_{j} d {R}_{2}^{*} + i d {ϕ}_{0} ) \} ] + {λ}_{3} d {m}_{0} = {\sum }_{j = 1}^{\# TE} [ - R e \{ \frac{{W}_{j}^{H}}{{m}_{0}} B \} ] \\ {\sum }_{j = 1}^{\# TE} [ - R e \{ {t}_{j}^{H} {W}_{j}^{H} {W}_{j} ( \frac{d {m}_{0}}{{m}_{0}} - {t}_{j} d {R}_{2}^{*} + i d {ϕ}_{0} ) \} ] + {λ}_{3} d {R}_{2}^{*} = {\sum }_{j = 1}^{\# TE} [ R e \{ {t}_{j}^{H} {W}_{j}^{H} B \} ] \\ {\sum }_{j = 1}^{\# TE} [ R e \{ {i}^{H} {W}_{j}^{H} {W}_{j} ( \frac{d {m}_{0}}{{m}_{0}} - {t}_{j} d {R}_{2}^{*} + i d {ϕ}_{0} ) \} ] + {λ}_{3} d {ϕ}_{0} = {\sum }_{j = 1}^{\# TE} [ - R e \{ {i}^{H} {W}_{j}^{H} B \} ]$$

Appendix C

The threshold, T, needs to be selected such that only the unreliable voxels in the data have their noise weighting reduced. As such, if T is smaller than the optimal value, then some of the reliable voxels would be penalized, resulting in the loss of data, but if T is larger than the optimal value, then some unreliable voxels would not be penalized, allowing these voxels to continue to generate artifacts in the solution. The attenuation factor, R, determines how much the noise weightings in voxels, that were picked up by a given T, are reduced. Large R could cause voxels to be over-penalized, leading to the loss of data, and small R could result in insufficient noise weighting reduction in unreliable voxels and insufficient suppression of artifacts. The current implementation of MERIT uses the parameters T=6σ, where σ is the standard deviation of the two-norm of the data-term residual, p, therefore, assuming that ρ is a Gaussian distribution, 0.3% of the voxels are penalized by MERIT at every GN iteration. In practice, however, it is unlikely that precisely 0.3% of voxels in every GN iteration contain model errors. Furthermore, the current MERIT implementation uses R=2, such that the noise weighting in the selection voxels are reduced by a factor of

$${( \frac{{ρ}_{i}}{T} )}^{2} ,$$

but there is no guarantee that this reduction factor will always return a more appropriate noise weighting. Implementation of MERIT can be found in the MEDI opensource toolbox

### 1.4 Results

### Simulation

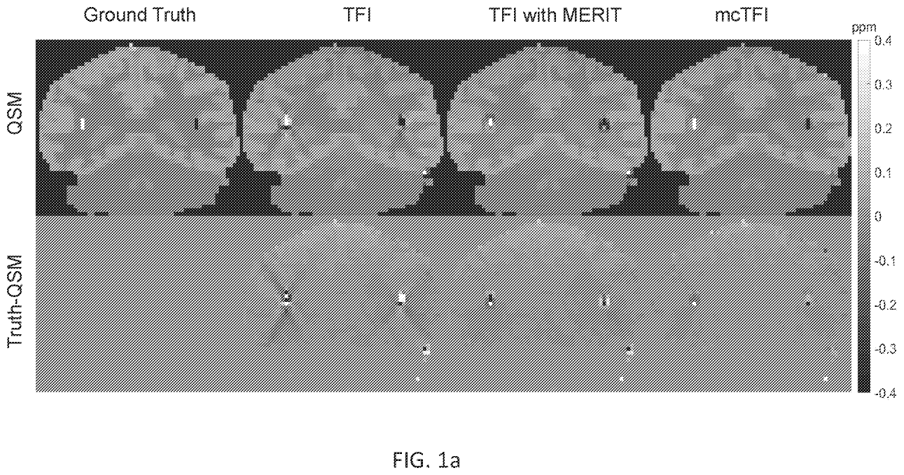

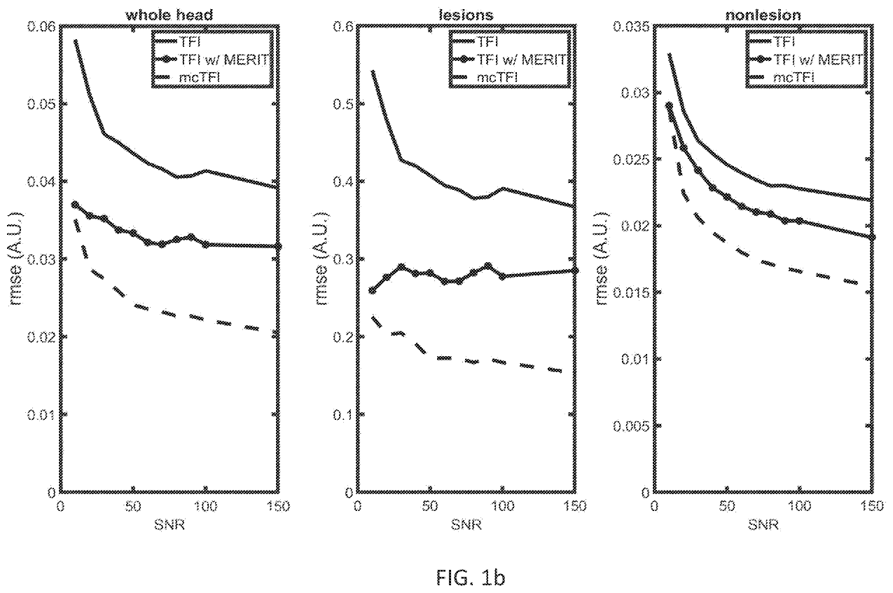

FIGS. 1a-1b show the comparison between TFI, TFI+MERIT, and mcTFI in a simulated brain that contains a calcified lesion and an ICH. As shown in FIG. 1a (SNR=100), TFI reconstructed QSM contains severe streaking artifacts that originated from the two lesions. The streaking artifacts were suppressed with the usage of MERIT, but the lesions susceptibilities were underestimated and the lesions themselves appeared enlarged. On the other hand, the mcTFI QSM contains no streaking artifacts, the lesion susceptibilities were in reasonable agreement with ground truth, and the lesions were not enlarged. FIG. 1B showed the mean RMSE over the 10 repeats at different SNR levels, and mcTFI results consistently resulted in better RMSE than the TFI and TFI+MERIT results.

### QSM Challenge 2.0

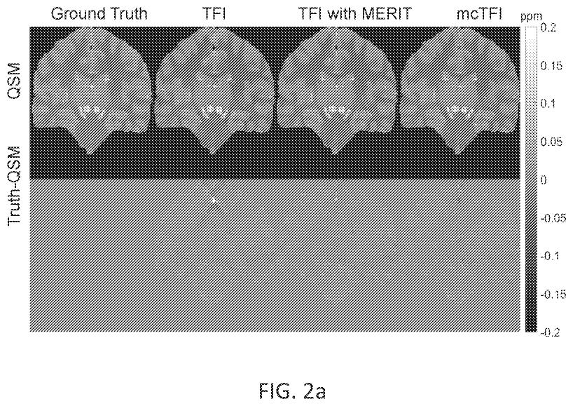

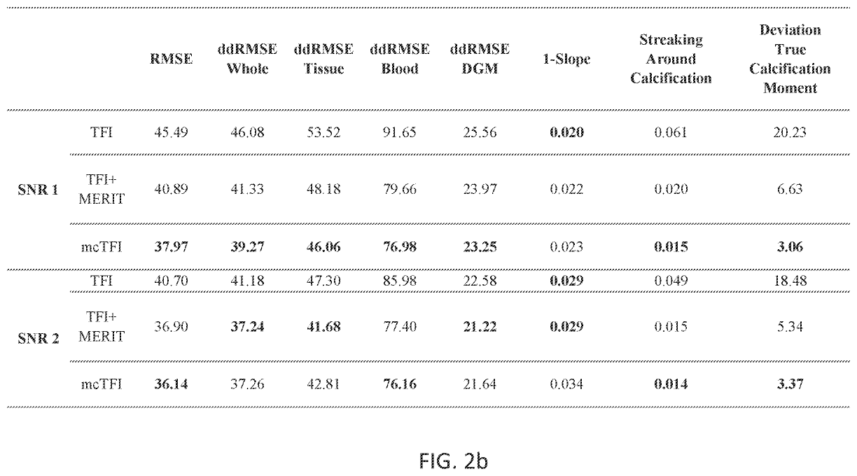

FIG. 2a shows the QSM reconstructed using TFI, TFI+MERIT, and mcTFI in one of the datasets provided by QSM Challenge 2.0. The TFI result contains streaking artifacts that originate from the calcified lesion. The streaking artifact was suppressed using MERIT, but the lesion size appeared larger than ground truth. The QSM from mcTFI contains the least streaking artifacts from the lesion and the lesion size is not enlarged compared to the ground truth. The QSM from the three methods is otherwise similar outside of the calcified lesion and streaking artifacts. This is also demonstrated in FIG. 2b, where the RMSE and streaking artifact measurements in TFI+MERIT and mcTFI are much better than that in TFI, because both TFI+MERIT and mcTFI results contain minimal streaking artifacts. But between TFI+MERIT and mcTFI, the latter produced more accurate calcification moment measurement compared to the ground truth.

In Vivo Brains with Hemorrhages

The image quality scores of the reconstructed hemorrhages for TFI, TFI+MERIT, and mcTFI were respectively 2.44±0.62, 1.78±0.35, and 1.11±0.21. There was a significant difference between the scores of TFI and TFI+MERIT (p=0.002), a significant difference between the scores of TFI and mcTFI (p<0.001), and significant difference between the scores of TFI+MERIT and mcTFI (p<0.001).

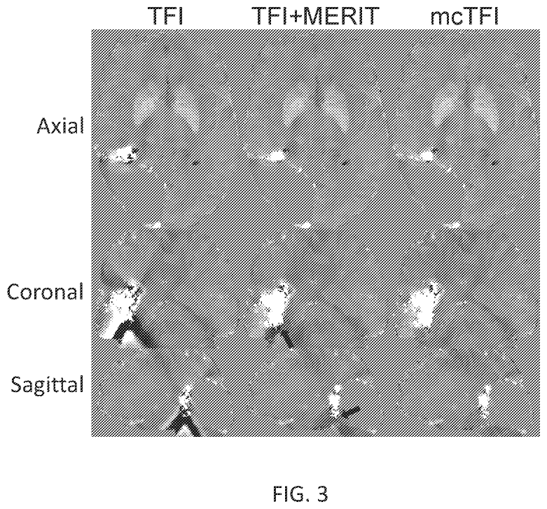

FIG. 3 shows the QSM reconstructions in a representative ICH brain presented in the three major planes. Severe streaking artifacts coming from the hemorrhage were observed in the TFI result; these streaking artifacts were significantly reduced in TFI+MERIT, but not completely suppressed (arrow). In mcTFI, the hemorrhage streaking artifacts appeared to be nearly suppressed.

### In Vivo Healthy Brains at 7T

The image quality scores for TFI, TFI+MERIT, and mcTFI were 2.86±0.24, 2.29±0.41, and 1.29±0.041, respectively. There was no significant difference between the scores of TFI and TFI+MERIT (p=0.102), a significant difference between the scores of TFI and mcTFI (p=0.002), and significant difference between the scores of TFI+MERIT and mcTFI (p=0.012).

FIGS. 10a-10d show the QSM reconstructions in a representative brain scanned at 7T. In case 1, significant artifacts in the prefrontal cortex area were observed in the TFI and TFI+MERIT results (arrows) while the proposed mcTFI effectively suppressed this artifact.

### 1.5 Further Description and Conclusions

QSM computes the susceptibility map from a susceptibility field, typically with the field first estimated by fitting a signal model to the acquired GRE images. The fitting errors and the non-zero mean Gaussian noise in the resulting fitted susceptibility field propagates through the subsequent dipole inversion step, causing artifacts in the final susceptibility map. The proposed mcTFI method computes the susceptibility map directly from the multiecho complex GRE images, while minimizing errors in field estimation and retaining the Gaussian noise property in the data term. The mcTFI method was compared with TFI (without and with MERIT), and mcTFI demonstrated both quantitative and qualitative improvements over TFI in simulations, brains with ICH, and healthy brains scanned at 7T.

The proper biophysical modeling is important for parameter extraction in quantitative MRI. For field estimations in the presence of fat chemical shift, for example, by modeling the fat chemical shift as part of the unknowns, as opposed to assuming it to be a constant value, the quality, and accuracy resulting estimated field was substantially improved. Prior to this work, the susceptibility field was first estimated via various approximations to Eq. 3, where different assumptions to the noise property of the field were made in the different approximations. For example, the TFI approach here estimated the susceptibility field by solving Eq. 9 (a simplified version of Eq. 3), in which the magnitude component of the complex images was assumed to be noiseless. The proposed mcTFI, on the other hand, solves the signal equation directly, which is a better signal model with fewer approximations and simplifications.

Another benefit of fitting the susceptibility map directly to the complex data is that the assumed noise in mcTFI is that of the acquired data. A separate field estimation commonly simplifies and alters the noise property, contributing to potential model errors in the subsequent dipole inversion step, which is typically formulated as maximum likelihood estimation by minimizing the quadratic data fidelity term ∥Dχ−f∥₂², thereby causing artifacts in the final susceptibility map. A noise weighting term, w, needs to be introduced to the data fidelity term ∥w(Dχ−f)∥₂² to mitigate the model errors and suppress the artifacts, but the proper estimation of w remains a challenge. This noise property related errors are substantial in tissue lesions with large susceptibilities such as hemorrhages and at ultra-high field strengths such as 7T. Accordingly, mcTFI can significantly improve QSM performance for these applications, as demonstrated in this work.

Currently, for field estimation using Eq. 9, the noise weighting is calculated as the covariance matrix of the fit in Eq. 9, but it does not properly account for the noise properties, as demonstrated by the suboptimal TFI results in this work. Further retuning of w using MERIT allows better suppression of artifacts from these model errors. However, MERIT is a heuristic method and dataset-specific MERIT parameters are required to ensure that MERIT suppresses the artifacts without introducing new artifacts (see appendix C in 1.3 in the above for details regarding the parameters). Looking at the examples in this work, when the MERIT parameters caused over-penalization, then, the streaking artifacts were effectively suppressed, but to over-penalized noise weighting in the voxels in and around the two lesions, led to the solver to fit for the field around the lesions first and the size of the lesions in the resulting susceptibility map appeared larger. In contrast, by using a more accurate model in the data term, mcTFI results demonstrated a sufficient suppression of streaking artifacts while retaining the true size of the lesions. Furthermore, the over-penalization of MERIT likely causes the loss of data in TFI, resulting in less accurate susceptibility map reconstruction in comparison to mcTFI. Similar behaviors were also demonstrated where streaking artifacts were effectively suppressed using MERIT, but lesions appeared larger. On the other hand, mcTFI was able to suppress streaking artifacts, while avoiding the introduction of new artifacts. In exemplary case demonstrated here, MERIT failed to completely suppress all streaking artifacts because it did not sufficiently penalize the noise weighting in all problematic voxels; in contrast, the QSM reconstructed using mcTFI suppressed streaking artifacts around the hemorrhage.

The current implementation of mcTFI can be readily extended in several aspects to fully embody the inventions conceptualized here. Firstly, the nonlinear and nonconvex objective function in mcTFI, by definition, has multiple local minima, and, therefore, its solutions may be sensitive to the choice of initial guess. There is no guarantee that the initial guess strategies as used in this implementation are optimal. This may be overcome by an alternative implementation that estimates the field first. As an exemplary embodiment for modeling the measured complex data with consideration of noise in both magnitude and phase information at each echo time, we have the following formulation to estimate field and other model parameters from the measured complex signal at each echo time:

$$( {m}_{0} , {R}_{2}^{*} , {ϕ}_{0} , f ) = {arg min}_{{m}_{0} , {R}_{2}^{*} , {ϕ}_{0} , f} {\sum }_{j = 1}^{\# TE} {‖ {m}_{0} {e}^{- {t}_{j} {R}_{2}^{*}} {e}^{i {ϕ}_{0}} {e}^{i {t}_{j} 2 π f} - {S}_{j} ‖}_{2}^{2} + R ( {m}_{0} , {R}_{2}^{*} , {ϕ}_{0} , f ) \qquad [ 10 ]$$

Then susceptibility is determined by inverting the field using the robust initialization method of alpha a scaling, which may be set to be much smaller than 1. As an exemplary embodiment for modeling a phase at each echo time according to a phase factor determined by the calculated magnetic field and the alpha scaling, we have the following formula to estimate susceptibility from the magnetic susceptibility dipole convolution modeling of the modeled phase at each echo time:

$${y}^{*} = {arg min}_{y} {\sum }_{j = 1}^{\# TE} {‖ {m}_{0} {e}^{- {t}_{j} {R}_{2}^{*}} ( {e}^{{it}_{j} 2 π α f} - {e}^{{it}_{j} 2 π d * Py} ) ‖}_{2}^{2} + λ {‖ {M}_{G} ∇ Py ‖}_{1} + {λ}_{2} {‖ {M}_{c} P ( y - {\overline{y}}^{c} ) ‖}_{2}^{2} \qquad [ 11 ]$$

A very small α may be used to linearize the exponential function in Eq. 11 for speeding up the computation with one deconvolution for all echoes (separation of echo summation and deconvolution or convolution) and overcoming initialization problems. Dividing the determined magnetic susceptibility distribution by the scaling factor, the final QSM map is

$$χ = \frac{1}{α} P {y}^{*} .$$

The modeled signal amplitude m₀e^{-t}^{ⱼ}^{R*}^{₂} in Eq. 11 may be replaced by the measured signal amplitude 11Si II.

Thirdly, the zero-reference term from a region of known uniform susceptibility, ∥M_{c}P(y−y̅^{c})∥₂² as employed in QSM reconstruction Eqs. 6, 7 and 11, provides not only an absolute susceptibility value for cross-space (center) and time (longitudinal) studies when such uniform susceptibility value is known such as zero for cerebrospinal fluid, but also suppression of shadowing artifacts. An aspect of this invention is to expand the zero-reference term to regions of known small susceptibility contrasts for suppressing shadowing artifacts, while avoiding penalty in regions of high susceptibility contrasts. The susceptibility contrasts can be estimated in several means, including normalized magnitude images, an approximate QSM map, and R2* map. The shadowing artifacts, characterized by the complimentary magic angle pattern over the whole image volume, can be measured by the first non-zero moment, which is the second moment or the variance over a region if the region is distributed over the whole image volume. Such a region is not necessarily contiguous in space but is scattered over the whole image volume or the object to enable effective sampling of the variations caused by shadowing artifacts. Then shadowing artifacts reduction can be expressed as minimizing variance in the regions of low susceptibility contrasts. We exemplify this shadowing artifacts reduction using a feature based on R2* map in the following manner. The regions of various susceptibility contrasts are estimated from R2* map according to bins of similar R2* values, low bins corresponding to low R2* values. Voxels sharing a characteristic, such as belonging to a distribution, are binned together. A distribution can be a distribution of R2* values, such as characterized by a rectangular function. A distribution can also be a distribution in space, such as a distribution of voxels of cerebrospinal fluid, cortical gray matter or white matter. A bin may be distributed in the image volume in a localized manner and/or an extensively scattered manner. An implementation of penalty weighting on a region of susceptibility contrast is to use a sigmoid function as exploited in the recent work of automated adaptive preconditioner. Then, as an exemplary embodiment for identifying a region of interest in a tissue that has a low susceptibility contrast according to a characteristic feature such as low R2* values and adding a second regularization term associated with enforcing the known low susceptibility contrast in the region of interest in the tissue, in addition to the zero-reference term of the cerebrospinal fluid region,

Σ_{g} [symbol] ( [symbol] _{g})∥M_{g}(χ−χ̅^{g})∥₂²/|M_{g}|, [12]

where [symbol] _{g} is a group average of R2* for the g-th group, [symbol] a weighting such as a sigmoid function, M_{g} the binary mask for the g-th group with |M_{g}| number of voxels, χ̅^{g} the average of susceptibility value in the g-th group, and the summation is over regions of low susceptibility contrasts. For identifying regions of low susceptibility contrasts, we group voxels with similar R2* values into various groups g's, and calculate their representative R2* value, [symbol] _{g}, such as the mean or median R2* values. Each group is defined by a bin, such as of 1-Hz width in R2* value. An example weighting function is the sigmoid function,

$$( {g}_{} ) ∼ ( {σ}_{2} - {σ}_{1} ) \frac{1}{1 + {e}^{- ( {g}_{} - {s}_{1} ) / {s}_{2}}} + {σ}_{1} .$$

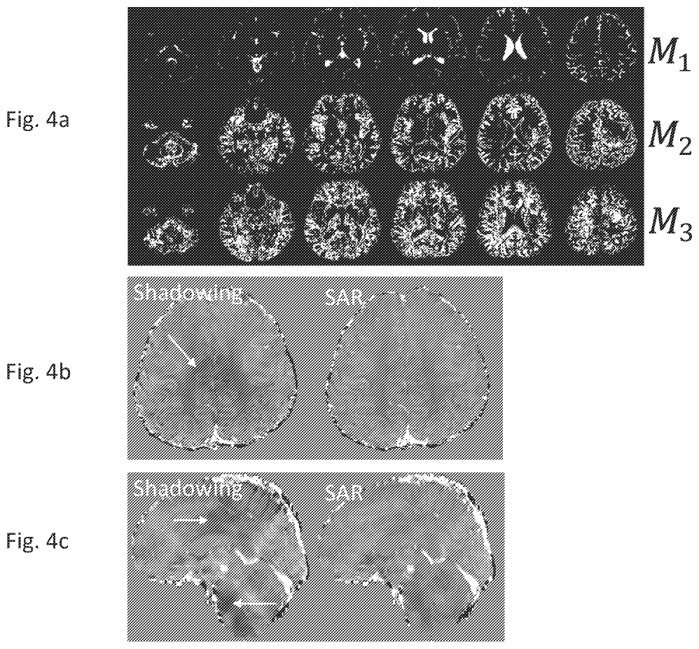

Here [σ₁, σ₂] control the output range and (s₁, s₂) control the shape. The choices of R2* binning and sigmoid curve are motivated by connections between R2* and susceptibility contrasts and by soft thresholding. [symbol] ( [symbol] _{g}) shall be high penalty (close to 1) in regions of low susceptibility contrasts (R2* close to 0), but little penalty (close to 0) in regions of high susceptibility contrasts (R2* above threshold). A step function approximating the sigmoid function as commonly used in deep learning may be used in Eq. 12. The penalties may be applied to the first two or three bins: first bin of the lowest R2* corresponds to most of cerebrospinal fluid in the whole brain, second bin of the second lowest R2* corresponds to most of cortical gray matter in the whole brain, and the third bin corresponds to a large portion of white matter next to cortical gray, as shown in FIG. 4a. Then the QSM reconstruction is formulated as fitting multiecho (number of echoes=N) complex data by penalizing streaking artifacts, such as using an L1 norm sparsity in locations where edges are not expected, and penalizing shadowing artifacts, such as using an L2 norm of low R2* bins.

$$( {m}_{0} , {R}_{2}^{*} , {ϕ}_{0} , χ ) = {arg min}_{{m}_{0} , {R}_{2}^{*} , {ϕ}_{0} , χ} {\sum }_{j = 1}^{N} {‖ {m}_{0} {e}^{- {t}_{j} {R}_{2}^{*}} {e}^{i {ϕ}_{0}} {e}^{i {t}_{j} 2 * χ} - {S}_{j} ‖}_{2}^{2} + {λ}_{1} {‖ {M}_{G} {∇}_{χ} ‖}_{1} + {λ}_{2} {\sum }_{g} ( {g}_{} ) {‖ {M}_{g} ( χ - {\overline{χ}}^{g} ) ‖}_{2}^{2} / \vert {M}_{g} \vert + {λ}_{3} R ( {m}_{0} , {R}_{2}^{*} , {ϕ}_{0} ) \qquad [ 13 ]$$

Preconditioning can be used to solve Eq. 13, as in Eqs. 4 & 11. An example of shadowing artifact reduction is illustrated in FIGS. 4b and 4c.

This low-contrast fidelity strategy of penalizing variance in the low contrast regions or bins, such as expressed by Σ_{g} [symbol] ( [symbol] _{g})∥M_{g}(χ−χ̅^{g})∥₂²/|M_{g}| can be employed to reduce shadowing artifacts in other image reconstructions where low contrast bins can be identified from existing or prior knowledge. For example, all MRI reconstructions can use this low-contrast-fidelity strategy to reduce shadowing artifacts, including various Bayesian QSM reconstructions, where low contrast regions can be identified on companying artifact-free images; all CT reconstructions can use this strategy to reduce streaking and shadowing artifacts, such as those originated from metal implants, where low contrast regions can be estimated from prior knowledge of metal implant geometry or from companying PET images.

Fourthly, the signal model in Eq. 5 can be further extended by considering the effects of deoxyheme iron on signal magnitude and phase. For example, the deoxyheme iron can be considered to have cylinder geometry and numerous with all directions in a voxel, while nonblood susceptibility sources can be considered as diffusely distributed in space. Then the magnitude signal at the j^{th} echo at echo time tⱼ with specific amount of deoxyheme iron as characterized by oxygenation Y is

$$\begin{matrix}{F}_{q B O L D} ( Y , v , {χ}_{n b} , {s}^{0} , {R}_{2} , {t}_{j} ) ≡ {s}^{0} {e}^{- {R}_{2} {t}_{j}} {e}^{- v f ( δ ω ( Y , {χ}_{n b} ) {t}_{j} )} g ( {t}_{j} ) , & [ 14 ] \\ where f ( δω \cdot TE ) {=}_{1} {F}_{2} ( [ - \frac{1}{2} ] ; [ \frac{3}{4} , \frac{5}{4} ] ; - \frac{9}{16} {( δω \cdot TE )}^{2} ) - 1 & \end{matrix}$$

with ₁F₂ the generalized hypergeometric function,

$$δ ω ( Y , {χ}_{n b} ) = \frac{1}{3} \cdot γ \cdot {B}_{0} \cdot [ Hct \cdot Δ {χ}_{0} \cdot ( 1 - Y ) + {χ}_{b a} - {χ}_{n b} ] ,$$

and g accounts for macroscopic contributions due to voxel sensitivity function. Here, y is the gyromagnetic ratio (267.513 MHz/T), B₀ is main magnetic field (3T in our study), Hct is hematocrit (0.357), Δχ₀ is the susceptibility difference between fully oxygenated and fully deoxygenated blood (4π××0.27 ppm), χ_{ba} is purely oxygenated blood susceptibility (−108.3 ppb). χ_{nb} non-blood susceptibility, and v the vein blood volume fraction. This forms the deoxyheme-iron modeled signal magnitude. The corresponding phase at the jth echo time with deoxyheme iron effects is

$$\begin{matrix}{ϕ}_{0} + {t}_{j} d * {χ}_{QSM} ( Y , v , {χ}_{nb} ) , where & [ 15 ] \\ {χ}_{QSM} ( Y , v , \text{} {χ}_{nb} ) ≡ [ \text{} \frac{( {χ}_{ba}}{a} + {ψ}_{Hb} \cdot {Δχ}_{Hb} \cdot ( - Y + \frac{1 - ( 1 - a ) \cdot {Y}_{a}}{a} ) ] \cdot v + ( 1 - \frac{v}{a} ) \cdot {χ}_{nb} . & \end{matrix}$$

Here a is the vein volume fraction in total blood volume and is assumed to be constant (0.77), ψ_{Hb} the hemoglobin volume fraction (0.0909 for tissue and 0.1197 for vein), Δχ_{Hb} the susceptibility difference between deoxy- and oxy-hemoglobin (12522 ppb). Eqs. 14 & 15 exemplify a modeled complex signal with a consideration of deoxyheme-iron effects on signal magnitude and phase at each echo time. By extending the signal model in Eq. 6 using Eqs. 14 & 15, we can formulate an optimization problem for determining

$$OEF = 1 - \frac{Y}{{Y}_{a}}$$

with Yₐ the arterial oxygen saturation (0.98) as

$${arg min}_{Y , v , {χ}_{nb} , {s}^{0} , {R}_{2}} {\sum }_{j = 1}^{N} {‖ {F}_{qBOLD} ( Y , v , {χ}_{n b} , {s}^{0} , {R}_{2} , {t}_{j} ) {e}^{i {ϕ}_{0}} {e}^{i {t}_{j} a * {χ}_{QSM} ( Y , v , {χ}_{n b} )} - {S}_{j} ‖}_{2}^{2} + λ R ( Y , v , {χ}_{n b} , {s}^{0} , {R}_{2} ) \qquad [ 16 ]$$

All regularizations considered before can be included in R(Y, v, χ_{nb}, s⁰, R₂). Coordinate descent method can be used to solve Eq. 16, as exemplified in Eqs. 7 & 8. Alternatively, the dipole deconvolution may be solved first as quantitative susceptibility mapping and then the inverse problem in the above Eq. 16 is solved without spatial dipole deconvolution.

Finally, an increase of model complexity in mcTFI also comes with an increase in computation time; the Alternating Direction Method of Multipliers (ADMM), which is a much faster algorithm, may be used efficiently solve the objective function in mcTFI.

In summary, this work provided the mcTFI method to directly estimate the susceptibility map from the GRE images, which is a more appropriate way to model the signal noise and bypass any errors from a separate field estimation. Compared to TFI, the provided mcTFI demonstrated improvements in susceptibility map reconstruction, with reduced streaking artifacts and improved performance in regions with low SNR.

### 2 Deep Neural Network (DNN) for Solving Inverse Problems in Imaging: Supervised Training, Unsupervised Training, and No Training

In this section, we describe solving optimization problems in QSM and other inverse problems such as fat water separation using deep neural network

### 2.1 Introduction

R2* corrected water/fat separation estimating fat, water and inhomogeneous field from gradient-recalled echo (GRE) is a necessary step in quantitative susceptibility mapping to remove the associated chemical shift contribution to the field. Several algorithms, including hierarchical multiresolution separation, multi-step adaptive fitting, and T2*-IDEAL, have been proposed to decompose the water/fat separation problem into linear (water and fat) and nonlinear (field and R2*) subproblems and solve these problems iteratively. Water/fat separation is a nonlinear nonconvex problem that requires a suitable initial guess to converge to a global minimum. Multiple solutions including 2D and 3D graph-cuts and in-phase echo-based acquisition have been proposed to generate an initial guess. The performances of these methods are dependent on the assumptions inherent in these methods, including single species dominant voxels, field smoothness, or fixed echo spacing to generate a suitable initial guess and avoid water/fat swapping.

Recently, deep neural networks (DNN) have been used to perform water/fat separation using conventional supervised training of DNN with reference data (STD). This STD water/fat separation method does not require an initial guess with the potential use of fewer echoes to shorten the scan time, or improve SNR with the same scan time, and lessen dependency on acquisition parameters compared to current standard approaches. However, the training of STD requires reference fat-water reconstructions (labels), which can be challenging to calculate as discussed above.

In this work, we investigate an unsupervised training of DNN (UTD) method that uses the physical forward problem in T2*-IDEAL as the cost function during conventional training without a need for reference fat-water reconstructions (no labels). We further investigate no-training DNN (NTD) method using a cost function similar to that in unsupervised to reconstruct fat-water images directly from a single dataset. We compare the results of the STD, UTD, and NTD methods with current T2*-IDEAL method.

### 2.2 Materials and Methods

### Water/Fat Separation T2*-IDEAL

Water/fat separation and field estimation is a nonconvex problem of modeling multiecho complex GRE signal (S) in terms of water content (W), fat content (F), field (f) and R*₂ decay per voxel:

$$( W , F , f , {R}_{2}^{*} ) = {arg min}_{W , F , f , {R}_{2}^{*}} {\sum }_{j = 1}^{N} {‖ {S}_{j} - {e}^{- {R}_{2}^{*} {t}_{j}} {e}^{- i 2 π {ft}_{j}} ( W + F {e}^{- i 2 π {v}_{F} {t}_{j}} ) ‖}_{2}^{2} , \qquad [ 17 ]$$

where N refers to the number of echoes, Si is the GRE signal S at echo time tⱼ with j=1, . . . , N, and v_{F} is the fat chemical shift in a single-peak model. T2*-IDEAL solves Eq. 17 by decomposing into linear (W, F) and nonlinear (f, R*₂) subproblems. With an initial guess for f and R*₂, the linear subproblem for W and F can be solved. With updated W, F, the nonlinear subproblem for f and R*₂ is linearized through first order Taylor expansion and solved using Gauss-Newton optimization. These steps are repeated until convergence is achieved. In this study, initial guesses for the field f and R*₂ decay were generated using in-phase echoes. The subsequent solutions to Eq. 17 resulted in the reference reconstructions Ψ_{REF}(S)={W, F, f, R*₂}.

### Supervised Training DNN (STD) Water/Fat Separation

In this work, we adapted the approaches in recent works and making W, F, f, and R*₂ the target output of the network. A U-net type network Ψ(Sⁱ; θ) with network weights θ is trained on M training pairs {Sⁱ, Ψ_{PREF}(Sⁱ)}, where Sⁱ and Ψ_{PREF}(Sⁱ)={W, F, f, R*₂}, are the input complex gradient echo signal and corresponding reference T2*-IDEAL reconstruction (reference), respectively, for the ith subject in the training dataset. The cost function for training the DNN is given by:

$${E}_{STD} = \frac{1}{2} {\sum }_{i = 1}^{M} {‖ {Ψ}_{R E F} ( {S}^{i} ) - Ψ ( {S}^{i} ; θ ) ‖}_{2}^{2} . \qquad [ 18 ]$$

### Unsupervised Training DNN (UTD) Water/Fat Separation

In the proposed method, termed unsupervised, we seek to use deep learning framework to calculate W, F, f, and R*₂ without access to reference reconstructions (labels). This is done by using the physical forward problem in Eq. 17 as the cost function during training. This cost function for training the DNN is given by:

$$\begin{matrix}{E}_{U T D} = \frac{1}{2} {\sum }_{i = 1}^{M} {\sum }_{j = 1}^{N} {‖ {S}_{j}^{i} - {\tilde{S}}_{j} ( Ψ ( {S}^{i} ; θ ) ) ‖}_{2}^{2} , & [ 19 ] \\ with {\tilde{S}}_{j} ( Ψ ) = {e}^{- {R}_{2}^{*} {t}_{j}} {e}^{- i 2 π f {t}_{j}} ( W + F {e}^{- i 2 π {v}_{F} {t}_{j}} ) & \\ for Ψ = \{ W , F , f , {R}_{2}^{*} \} . & \end{matrix}$$

### No-Training DNN (NTD) Water/Fat Separation

Recently, a single test data is used to update DNN weights in deep image prior and fidelity imposed network edit. This inspires the idea that DNN weights θ* initialized randomly may be updated on a single gradient echo data set S to minimize the cost function in Eq. 17 in a single data set S.

$${E}_{NTD} = \frac{1}{2} {\sum }_{j = 1}^{N} {‖ {S}_{j} - {\tilde{S}}_{j} ( Ψ ( {S}_{j} ; θ ) ) ‖}_{2}^{2} \qquad [ 20 ]$$

The resulting network weights are specific to the data S, and the resulting output Ψ(S; θ*) can be taken as the water/fat separation reconstruction of S. In contrast to the above STD and UTD that involve conventional training data, no training is required here, the cost function is the same as that in the unsupervised training. Therefore, this method is referred to as no-training DNN (NTD).

### Network Architecture

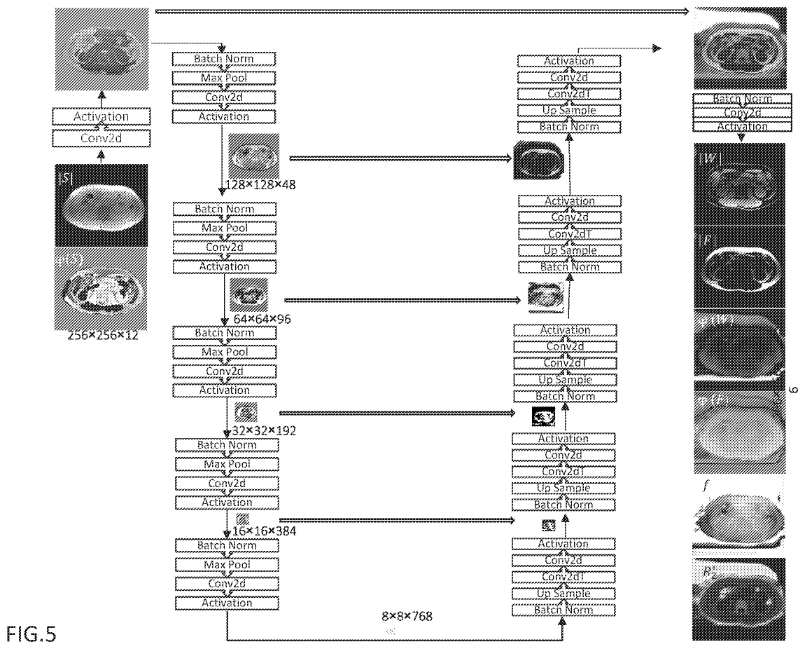

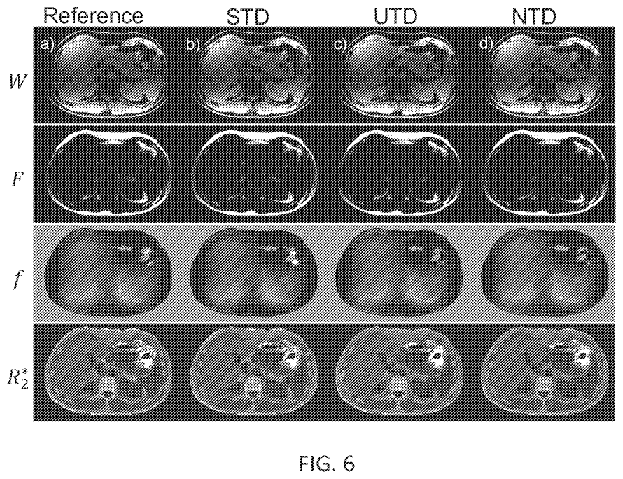

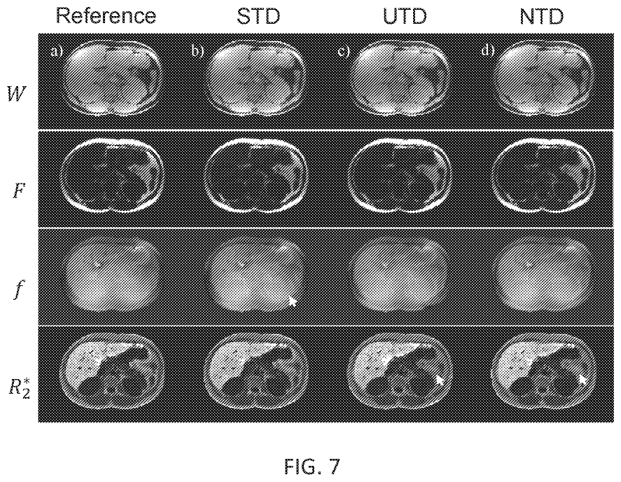

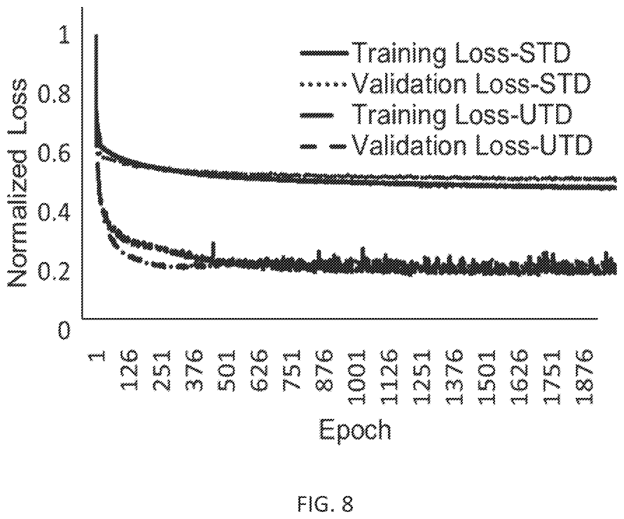

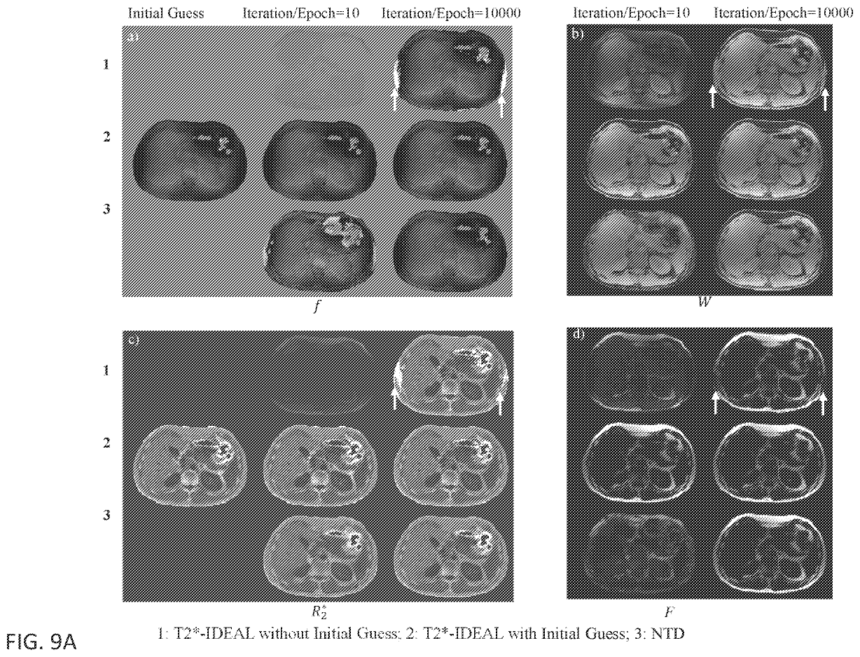

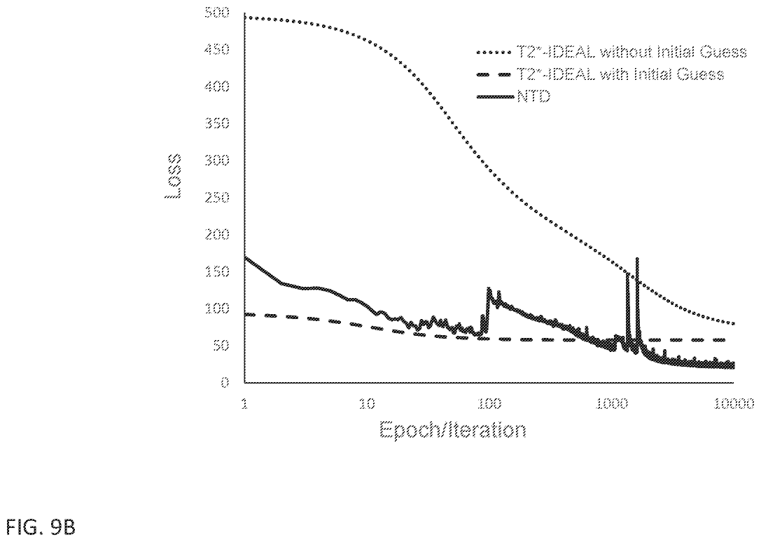

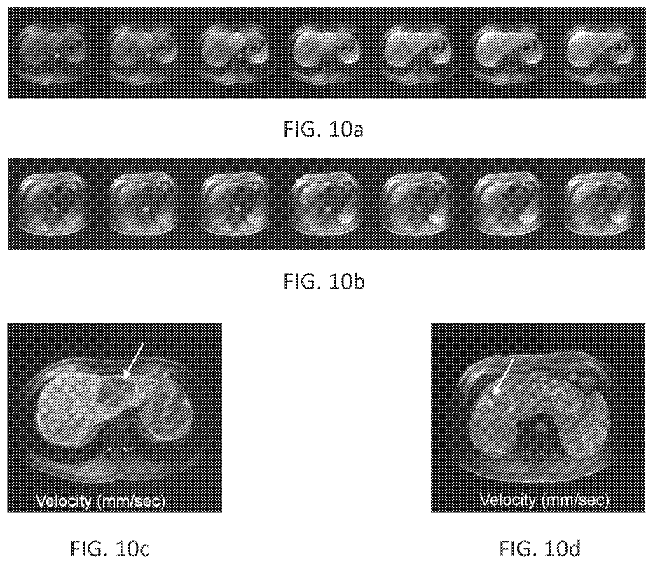

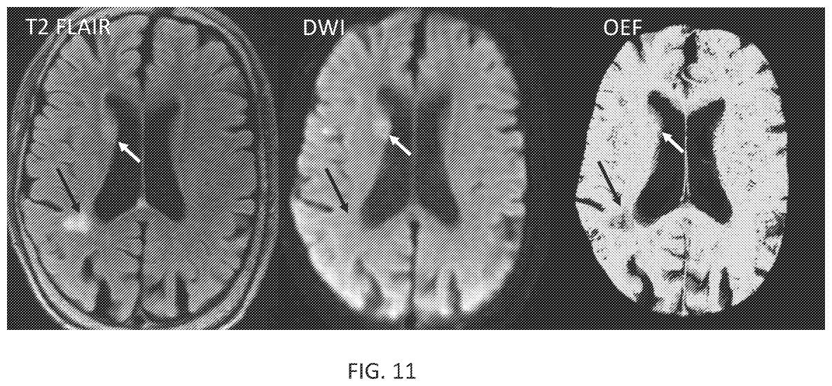

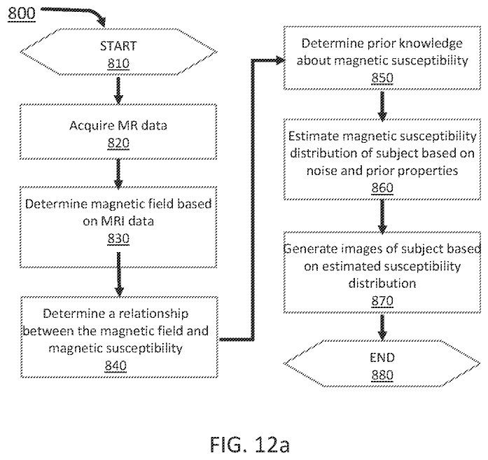

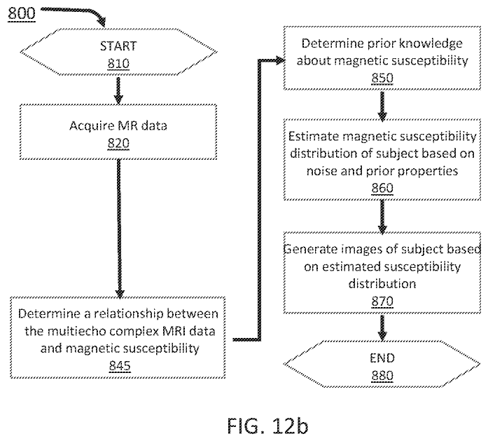

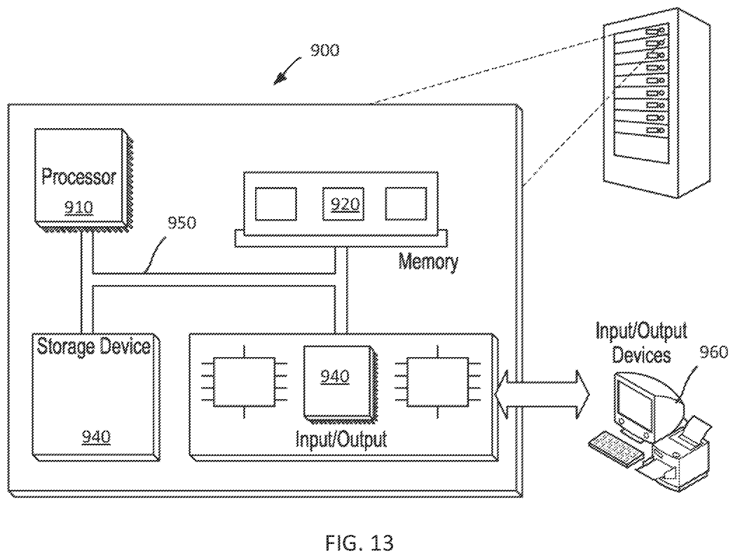

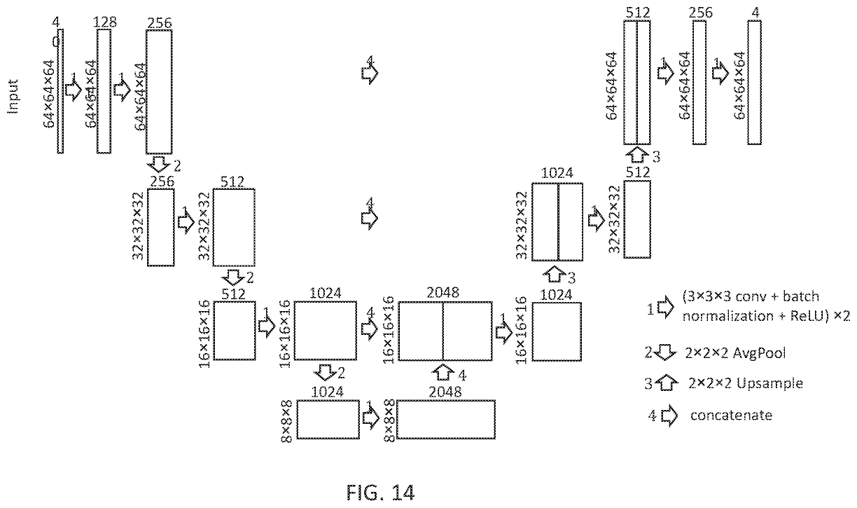

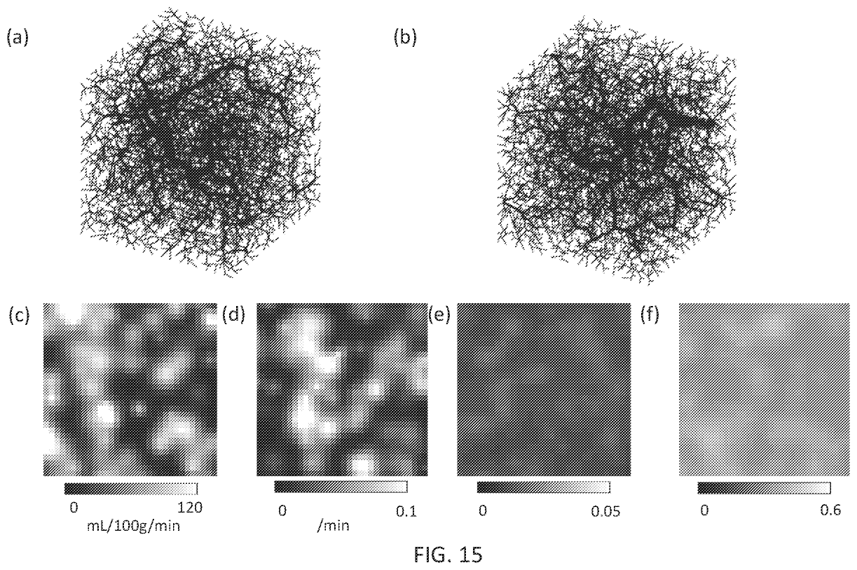

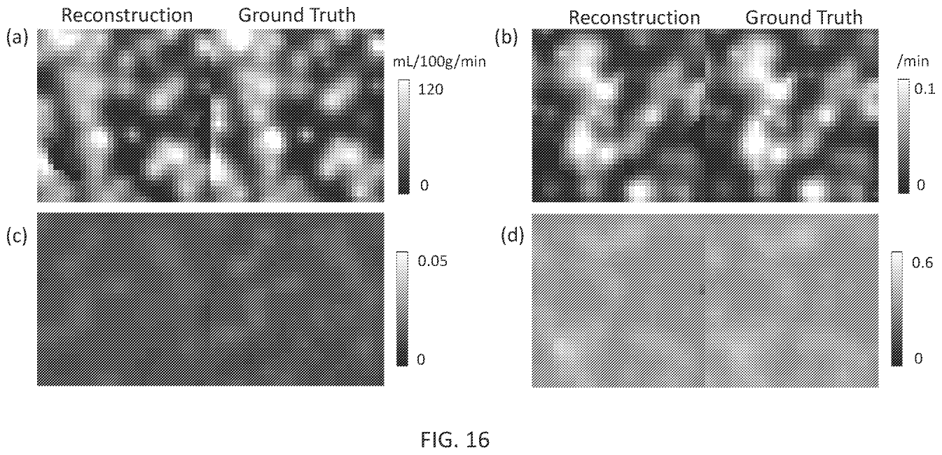

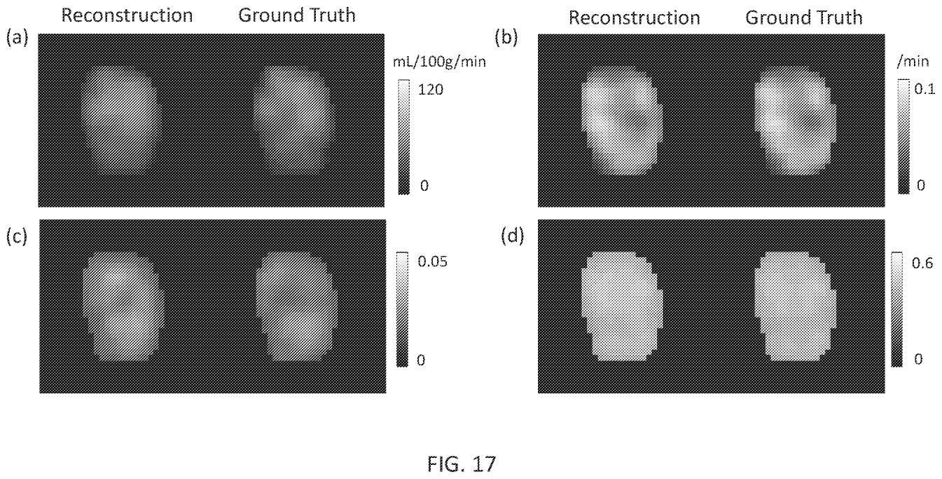

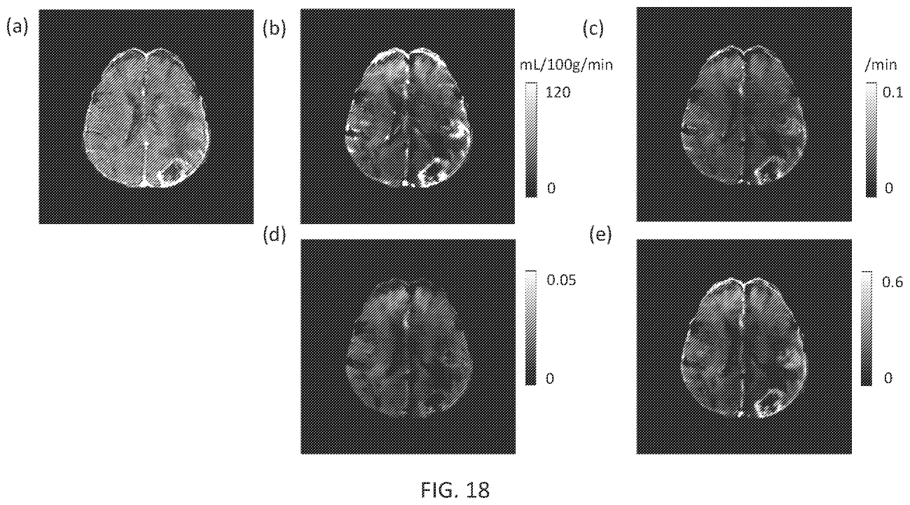

The network Ψ(Sⁱ; θ) was a fully 2D convolutional neural network with encoding and decoding paths (FIG. 5). The encoding path included repeated blocks (n=5), each comprises convolution (2×2), activation function (Sigmoid), batch normalization and max pooling (2×2). The decoding path with repeated blocks (n=5) has similar architecture except max pooling is replaced with deconvolution and upsampling along with concatenation of corresponding feature maps with the encoding path. The last bock comprises convolution with linear activation function. The input to the network comprised 2N channels (the magnitude and phase of the gradient echo signal for each echo). The output of the network comprised 6 channels (the magnitude and phase of the water and fat images, plus the field map and R2*). FIG. 11 shows the network architecture exemplified by a U-net, representative input and output images for a test set in UTD method, along with outputs of intermediate layers. These show how learned features at different levels transform the input data into the final output images. Note that arrows indicate concatenation of encoder and decoder layer outputs with the same feature maps. Training was performed using the ADAM optimizer.

### Data Acquisition

Data was acquired in healthy volunteers (n=12) and patients (n=19), including thalassemia major (n=11), polycystic kidney disease (n=7), and suspected iron overload (n=1). The study was approved by the Institutional Review Board and written informed consent was obtained from each participant.

Two 1.5T GE scanners (Signa HDxt, GE Healthcare, Waukesha, WI) with 8-channel cardiac coil were used to acquire data. The healthy subjects were imaged on both scanners using identical protocols. Patients were scanned on one scanner, selected at random, using the same protocol. This protocol contained a multiecho 3D GRE sequence with the following imaging parameters: number of echoes=6, unipolar readout gradients, flip angle=5°, ΔTE=2.3 msec, TR=14.6 msec, acquired voxel size=1.56×1.56×5 mm³, BW=488 Hz/pixel, reconstruction matrix=256×256×(32-36), ASSET acceleration factor=1.25, and acquisition time of 20-27 sec.

### Experiments and Quantitative Analysis

Multiple experiments were performed to assess the performance of DNN in water/fat separation and how supervised (STD), unsupervised (UTD), and no-training (NTD), compare against T2*-IDEAL reference method. The network architecture for Ψ(Sⁱ; θ) was identical between the three DNN methods. Parameters in STD and UTD include, number of epochs 2000, batch size 2, learning rate 0.001 for STD and 0.0001 for UTD. The learning rates were experimentally found to produce optimal results. In NTD, parameters include, number of epochs 10000, batch size 2, and learning rate 0.0001.

The supervised and unsupervised DNN were trained on the combined data set of healthy subjects (2 scans each) and patients. This data of n=43 scans was split into testing (256×256×61×6) and training (256×256×1522×6) with 80% of the latter used to training and the rest for validation. The weights corresponding to the lowest validation loss during training was selected as the optimal weights to be used during testing. Training time in each epoch was ˜60 seconds. The testing data comprised of two datasets, one healthy subject and one patient with iron overload. In the test data, ROIs were drawn on the proton density fat fraction map

$$PDFF = \frac{\vert F \vert}{\vert F \vert + \vert W \vert} ,$$

the field and R2* in several regions including liver, adipose and visceral fat, aorta, spleen, kidney, vertebrae. The ROI measurements for each DNN method were compared with those measured on the reference T2*-IDEAL maps using correlation analysis. Reference generation with T2*-IDEAL was performed on CPU (Intel, i7-5820 k, 3.3 GHz, 64 GB) using MATLAB (MathWorks, Natick, MA) and all DNN trainings were performed on GPU (NVIDIA, Titan XP GP102, 1405 MHz, 12 GB) using Keras/TensorFlow.

FIG. 6 compares the network output results in the healthy test subject for STD (FIG. 12b), UTD (part c) of FIG. 6, NTD (part d) of FIG. 6) with the reference T2*-IDEAL reconstruction (part a) of FIG. 6). The correlation plots with T2*-IDEAL for STD, UTD, and NTD in Proton Density Fat Fraction (PDFF) (F), field (f) and R2* show excellent agreement with slopes close to 1 and R²≥0.98. While in this case water and fat images agree well with reference, field and R2* shows poor qualitative and quantitative agreement with the reference. This suggests while both formats (real/imaginary or magnitude/phase) have identical information content, spatial and temporal distribution of information differs from one format to another which makes the learning task easier in the latter case.

FIG. 7 compares the network output results in the moderate iron-overload test patient for STD (part b) of FIG. 7), UTD (part c) of FIG. 7), NTD (part d) of FIG. 7) with the reference T2*-IDEAL reconstruction (FIG. 3a). Very good qualitative agreement in both contrast and details is observed among these outputs. There were only marginal deviations in the field of STD in the anterior part of the abdomen near the kidney (arrow in part b) of FIG. 7), and marginal differences in R2* maps among the reference and STD, in comparison with UTD (arrow in part c) of FIG. 7) and NTD (arrow in part d) of FIG. 7). Correlation analysis comparing PDFF (F), field (f) and R2* show excellent agreement with slopes close to 1 and R2 ≥0.96.

FIG. 8 shows the normalized training and validations losses for the STD and UTD methods. While validation loss for the STD method is initially (epoch <500) lower than the training loss it becomes larger at later epochs while the opposite trend is observed for UTD. The total training time for both methods was similar (˜33 hrs each).

FIGS. 9A-9B show a comparison of NTD with T2*-IDEAL. The T2*-IDEAL cost function per iteration is shown in the same graph as the NTD reconstruction cost per epoch. In the first row, T2*-IDEAL results of field (part a) of FIG. 9A), water (part b) of FIG. 9A), R2* (part c) of FIG. 9A) and fat (part d) of FIG. 9A) shows partial failure in several regions (arrows) when field and R2* were initialized with zeros. In the second row, the corresponding images show the result of T2*-IDEAL when using a proper initialization (the field and R2* obtained from the in-phase echoes in this case). The third row shows the proposed NTD results. The generated maps when using 10000 epochs are close the successful T2*-IDEAL result (2^{nd} row) without a need for an initial guess. The corresponding T2*-IDEAL costs and NTD reconstruction loss are shown in FIG. 9B. Without initialization, T2*-IDEAL requires more iterations (10000) compared to T2*-IDEAL with initialization (100). The proposed NTD method requires 10000 epochs for convergence. Computation time for each iteration and each epoch was similar (˜2 seconds).

### 2.4 Further Description and Conclusion

Our results demonstrate the feasibility of an unsupervised deep neural network (DNN) framework with conventional training and without training to solve the water/fat separation problem. The proposed unsupervised training DNN (UTD) method does not require reference images as in supervised training DNN (STD), allowing the use of DNNs for training data that are unlabeled but for which physical model is known. The no-training DNN (NTD) method further allows using DNN reconstruction of a single data set (subject) for which a physical model is known.

For the nonlinear nonconvex water/fat separation problem, the reference T2*-IDEAL method used traditional gradient descent optimization and is dependent on the initial guess. This problem may be alleviated using deep learning, as long as the labeled training data is sufficiently large to capture test data characteristics. Labeled data may be difficult to obtain, in as water/fat separation problems. Unlabeled data are easier to obtain, but still it is difficult to ensure that training data do not lack test data characteristics. Accordingly, reconstruction directly from a test data without training is desirable, as in the reference T2*-IDEAL but without its dependence on initialization. This can be achieved using the proposed NTD.

The NTD method can overcome the initialization-dependence in traditional gradient descent based nonconvex optimization begs some intuitive explanation or interpretation, though rigorous explanation is currently not available. The cost function in DNN is minimized by adjusting network weights through backpropagation, which is achieved through iterative stochastic gradient descent (SGD). Perhaps SGD allows escaping local traps encountered in traditional gradient descent and the intensive computation in updating network weights facilitates some exhaustive search for a consistent minimum. The network weights updating on a single test data may converge as demonstrated in fidelity imposed network edit and in deep image prior. Our data suggests that NTD can converge to a consistent minimum without initialization-dependence for the nonlinear nonconvex water/fat separation problem.

While some image details in STD, UTD, and NTD methods were different from reference images, quantitative measurements in the liver include ROI measurements of several voxels and regions which is less dependent on image details. The network architecture can be further optimized for this problem. For instance, we found changing activation function (Relu to Sigmoid) significantly improved field and R2* results in unsupervised training while not much change was observed in water and fat maps. Since field and R2* have a nonlinear relationship with respect to input complex signal while water and fat are linear, there is possibility of designing task-specific blocks to learn the linear and nonlinear mapping during training for further improvement and potentially decreasing the number of learned weights.

The exemplary implementation in this study can be extended and improved readily in the following manners. A ready extension is to apply the DNN methods, including STD, UTD and NTD, to solve QSM inverse problem. For example, the UTD method Eq. 19 can be updated with the signal model in Eq. 5 for QSM reconstruction Ψ=(m₀, R*₂, ϕ₀, χ): \tilde{S}ⱼ(Ψ)=m₀e^{-t}^{ⱼ}^{R*}^{₂}e^{iϕ}^{₀}e^{it}^{ⱼ}^{ω}^{₀}^{Dχ}. The regularization terms for penalizing streaking and shadowing artifacts and for denoising as in Eq. 13 can be added to the cost function in DNN as in Eq. 19. An example QSM reconstruction using DNN is:

$${E}_{UTD} = \frac{1}{2} {\sum }_{i = 1}^{M} {\sum }_{j = 1}^{N} {‖ {S}_{j}^{i} - {\tilde{S}}_{j} ( Ψ ( {S}^{i} ; θ ) ) ‖}_{2}^{2} + {λ}_{1} {‖ {M}_{G} ∇ χ ‖}_{1} + {λ}_{2} {\sum }_{g} 𝒮 ( {ℛ}_{g} ) {‖ {M}_{g} ( χ - {\overline{χ}}^{g} ) ‖}_{2}^{2} / \vert {M}_{g} \vert + {λ}_{3} R ( {m}_{0} , {R}_{2}^{*} , {ϕ}_{0} ) \qquad [ 21 ]$$

Then Eq. 21 describes unsupervised training for deep learning reconstruction of QSM using multiecho complex data as detailed in the above section 2. Similarly, Eqs. 18 & 20 can be extended in the above manner for QSM reconstruction using deep learning.

Another extension is to apply the DNN methods, including STD, UTD an NTD, to solve transport inverse problem, quantitative transport mapping (QTM), from multiple timeframes of time-resolved tracer passage through tissue. DNN methods offer particular advantages in denoising poorly conditioned transport inversion problem of targeted tracers including targeted theranostic agents and targeted agents. The tracer concentration in a voxel r at time t, c(r, t), comprised of an arterial flow or convection component with velocity u(r) and concentration cₐ(r, t), and a tissue bound component with concentration c₁(r, t). The transport equation of mass flux comprised of

∂ₜcₐ(r,t)=−∇cₐ(r,t)u(r)−k₁cₐ(r,t)+k₂c₁(r,t),

∂ₜc₁(r,t)=k₁cₐ(r,t)−k₂c₁(r,t), [22]

resulting in an example for the transport equation of tracer velocity-exchange model for the observed concentration as

∂ₜc(r,t)=−∇(1+T(k₁,k₂;t)*)⁻¹c(r,t)u(r). [23]

Here, the temporal convolution operation T(k₁, k₂; t) accounts for transiently binding or exchange effects characterized by exchange coefficients k₁, k₂ (exchange coefficients and maybe equal for some tracers) is defined as in solving the second part of the transport equation:

c₁(r,t)=T(k₁,k₂; t)*cₐ(r,t)=k₁e^{-k}^{₂}^{t}∫₀^{t}e^{k}^{₂}^{t}′Cₐ(r,t′)dt′=k₁e^{-k}^{₂}^{t}δₜΣᵢ₌₁ⁿe^{k}^{₂}^{δ}^{ₜ}ⁱcₐ(r,δₜi), [24]

which allows a straightforward sequential calculation of (1+T(k₁, k₂; t))⁻¹c(r,δₜi) from i=1, n with t=δₜn and δₜ is the imaging time frame. Then the observed signal ∂ₜcⁱ(r, δₜj)=Sⱼⁱ in the ith training subject is fitted to the transport equation physics model \tilde{S}ⱼ(Ψ)=−∇(1+T(k₁, k₂; δₜj)*)⁻¹c(r, δₜj)u(r), which forms the velocity and exchange coefficient modeled transport equation. The DNN methods can be applied to reconstruct Ψ=(k₁, k₂, u(r)). For example, the NTD method Eq. 20 can be updated as

$${E}_{Q T M} = \frac{1}{2} {\sum }_{j = 1}^{N} {‖ {S}_{j} - {\tilde{S}}_{j} ( Ψ ( S ; θ ) ) ‖}_{2}^{2} + + λ R ( {k}_{1} , {k}_{2} , u ( r ) ) \qquad [ 25 ]$$

Here the regularization R(k₁, k₂, u(n) can be sparsity formulated by L1 norm that are effective for denoising in image processing and reconstruction; other regularization may also be considered, including smooth regularization using L2 norm. The DNN approach to solving the inverse transport problem in determining transport parameters from the spacetime resolved tracer concentration data can also advantageously applied to other transport situations, including non-binding tracers with the transport equation simplified to ∂ₜc(r, t)=−∇c(r, t)u(r), non-binding diffusion tracers with the transport equation ∂ₜc(r, t)=−∇c(r, t)u(r)+∇D(r)∇c(r, t), and binding tracers with multiple compartments where the temporal convolution operator is extended with more exchange coefficients. The QTM problem Eq. 25 can be solved without using DNN as in Eq. 6. Noise propagation through the inversion can be overcome easily using DNN. Simulated data can be used to implement STD method, or unsupervised approached can be used to implement UTD method by minimizing E_{QTM} for all training cases. DNN training may also provide regularization implicitly without specific L1 or L2 formulation. An example of QTM processed velocity of the liver is illustrated in FIG. 10. The method of quantitative transport mapping described in the above can be applied to spacetime resolved tracer concentration images estimated from all imaging situations, including magnetic resonance imaging, such as dynamic contrast enhanced MRI, dynamic susceptibility contrast MRI, and multiple delay arterial spin labeling MRI, dynamic contrast enhanced computed tomography (CT), dynamic single photon emission computed tomography (SPECT), and dynamic positron emission tomography (PET).

And another extension is to apply the DNN methods, including STD, UTD and NTD, to solve the inverse problem of mapping oxygen extraction fraction (OEF) from multiecho gradient echo complex data. The OEF problem formatted in Eq. 16 can be solved using the DNN method with the following cost function: Ψ=(Y, v, χ_{nb}, s⁰, R₂), \tilde{S}ⱼ(Ψ)=F_{qBOLD}(Y, v, χ_{nb}, s⁰, R₂, tⱼ)e^{iϕ}^{₀}e^{it}^{ⱼ}^{d*χ}^{_{QSM}}^{(Y, v, χ}^{_{nb}}⁾,

$${E}_{O E F} = \frac{1}{2} {\sum }_{j = 1}^{N} {‖ {S}_{j} - {\tilde{S}}_{j} ( Ψ ( S ; θ ) ) ‖}_{2}^{2} ++ λ R ( Y , v , {χ}_{n b} , {s}^{0} , {R}_{2} ) \qquad [ 26 ]$$

DNN can be used to formulate regularization in training using synthetic data and/or in vivo data. DNN framework can be used to solve the inverse problem.

Alternatively, the multiecho phase images are processed using QSM according to Eq. 6 or Eq. 21 as described in the above. The multiecho magnitude images are processed according the model of deoxyheme iron in cylinders and other susceptibility sources are diffuse, i.e., the magnitude signal for the j^{th} echo at/echo time jΔTE is

|sⱼ|=F_{qBOLD}(Y,v,χ_{nb}, s⁰, R₂, jΔTE), [27]

For determining

$$OEF = 1 - \frac{Y}{{Y}_{a}}$$

with Yₐ the arterial oxygen saturation (0.98), the reconstruction problem from multiecho data is Ψ=(Y, v, χ_{nb}, s⁰, R₂):

\tilde{S}ⱼ(Ψ)=|sⱼ|=F_{qBOLD}(Y,v,χ_{nb}, s⁰, R₂,jΔTE)∀j=1, 2, . . . , N and

$${\tilde{S}}_{N + 1} ( Ψ ) = \sqrt{w} {χ}_{Q S M} ( Y , v , {χ}_{n b} ) = \sqrt{w} [ \frac{{χ}_{b a}}{a} + {ψ}_{H b} \cdot {Δχ}_{H b} \cdot ( - Y + \frac{1 - ( 1 - α ) \cdot {Y}_{a}}{α} ) ] \cdot v + ( 1 - \frac{v}{α} ) \cdot {χ}_{n b} , \qquad [ 28 ]$$

with w the weight accounting for different noise property from |sⱼ|·\tilde{S}_{N+1}(Ψ) forms the deoxyhem-iron modeled tissue magnetic susceptibility. DNN can be used to perform this reconstruction from multiecho magnitude signal Sⱼ ∀j=1, 2, . . . , N and QSM=S_{N+1}. For example, the NTD method Eq. 20 can be updated as

$${E}_{O E F} = \frac{1}{2} {\sum }_{j = 1}^{N + 1} {‖ {S}_{j} - {\tilde{S}}_{j} ( Y , v , {χ}_{n b} , {s}^{0} , {R}_{2} ) ‖}_{2}^{2} + + λ R ( Y , v , {χ}_{n b} , {s}^{0} , {R}_{2} ) \qquad [ 29 ]$$

Here the regularization R(Y, v, χ_{nb}, s⁰, R₂) can be sparsity formulated by L1 norm that are effective for denoising in image processing and reconstruction. The OEF problem defined by the cost function Eq. 29 can be solved without using DNN as in Eq. 6. Noise propagation through inversion can be overcome easily using DNN that is known to have denoising properties. Simulated data can be used to implement STD method, or unsupervised approached can be used to implement UTD method by minimizing E_{OEF} for all training cases. An example of OEF map of the brain is illustrated in FIG. 11. The approach in Eq. 26 with the complex signal of proper noise modeling may be robust.

In general, a ready improvement of the DNN exemplified in FIG. 5 is to replace 2D convolution with 3D convolution, add more layers, and use other network architectures and strategies. Another ready improvement for the STD and UTD trained DNN methods is to impose data fidelity on the network output by updating the network weights according to the test data and Eq. 20. This fidelity-imposed network edit overcomes the problem when test data deviates substantially from the characteristics of training data.

And another extension is to allow different R2* decays for water and fat in the water/fat separation and field estimation problem. As an example, Eq. 17 can be updated by modeling multiecho complex GRE signal (S) in terms of water content (W), fat content (F), field (f), R*_{2W} for water component decay and R*_{2F} for fat component decay:

$$( W , F , f , {R}_{2}^{*} ) = {argmin}_{W , F , f , {R}_{s}^{*}} {\sum }_{j = 1}^{N} {‖ {S}_{j} - {e}^{- i 2 π {ft}_{j}} ( {We}^{- {R}_{2 W}^{*} {t}_{j}} + {Fe}^{- {R}_{2 F}^{*} {t}_{j}} {e}^{- i 2 π {v}_{F} {t}_{j}} ) ‖}_{2}^{2} . \qquad [ 30 ]$$

Different R2* decay rates for fat and water components are important for certain biomedical imaging situations, such as a voxel of mixture of marrow fat and bone water. Then Eqs. 18-20 can be updated accordingly for the fat-water separation problem.

The above Eq. 30 for the fat-water signal model can be further extended by including a spectrum of chemical shift. For example, e^{-i2πv}^{_{F}}^{t}^{ⱼ} in Eq. 30 can be replaced with Σᵥwᵥe^{-i2πvt}^{ⱼ}, where wᵥ is the spectral distribution and Σᵥ, wᵥ=1.

While the input data is complex, the network only accepts real values and two separate input channels were used instead. This potentially can change the noise properties and suboptimal network performance which can be addressed by including complex networks for this purpose. While the network in both supervised (STD) and the unsupervised (UTD) methods were trained with specific acquisition parameters, it's possible to generalize by including more cases with different acquisitions parameters for training. Generation of reference images requires solving the T2*-IDEAL problem which can be challenging to calculate depending on acquisition protocol. The advantage of the proposed unsupervised methods is they only require complex input signal for training. While the NTD method requires a large number of epochs to converge which is computationally expensive, one could use a previously trained network (trained on data with the same imaging parameters) and update the weights for the new data set. Only one scanner manufacturer/model was used for this study and including additional models, manufacturers and field strengths will help to further generalize. There were limited number of cases used for training in STD and UTD methods in this study and including more cases will leverage network performance and reliability.

In summary, we demonstrated the feasibility of using unsupervised DNNs to solve the water/fat problem with very good agreement compared to reference images.

### 3 Device System Implementation

In some cases, one or more of the above quantitative susceptibility mapping techniques can be implemented using the process 800 shown in FIG. 12. As an example, the process 800 can be used to map the tissue magnetic susceptibility of a subject, such as a patient (or portion of a patient) or an experimental sample (e.g., an imaging phantom, a sample of material or tissue, and so forth).

In some implementations, the process 800 can be used to transform MRI signal data corresponding to a subject into susceptibility-based images that quantitatively depict the structure and/or composition of the subject. As an example, in some cases, the process 800 can be used to obtain MRI data corresponding a subject, and process this MR data to generate a quantitative susceptibility map of the subject. Using this quantitative susceptibility map, one or more susceptibility-based images of the subject can be generated and displayed to a user. The user can then use these images for diagnostic or experimental purposes, for example to investigate the structure and/or composition and/or function of the subject, and/or to diagnose various conditions or diseases based, and/or to treat various conditions or diseases based, at least in part, on the images. As the process 800 can result in susceptibility-based images having higher quality and/or accuracy compared to other susceptibility mapping techniques, implementations of the process 800 can be used to improve a user's understanding of a subject's structure and/or composition and/or function, and can be used to improve the accuracy of any resulting medical diagnoses or experimental analyses.

The process 800 begins by acquiring magnetic resonance (MR) data corresponding to a subject (step 810). In some cases, the MR data can correspond to a patient (e.g., the entirety of a patient or a portion of a patient, such as a particular portion to the patient's body). In some cases, the MR data can correspond to one or more samples (e.g., an imaging phantom, a sample of one or more materials, a sample of one or more types of tissue, and/or one or more other objects).

In some implementations, the MR data can be acquired using an MRI scanner using one or more suitable pulse sequences. As an example, in some cases, MR data can be acquired using a gradient echo sequence that acquires MR data at a single echo time or at multiple echo times (e.g., two, three, four, five, and so forth). Various scan parameters can be used. As another example, MR data can be obtained using a 3D multiecho spoiled gradient echo sequence on a 3T scanner (e.g., a GE Excite HD MR scanner) using a pulse sequence that samples at different echo times (TE) (e.g., 4 TEs, such as 3.8, 4.3, 6.3 and 16.3 ms) in an interleaved manner, and with following imaging parameters: repetition time (TR)=22 ms; voxel size=0.5×0.5×0.5 mm³, matrix size=256×64×64; bandwidth (BW)=±31.25 kHz; flip angle=15°. As another example, MR data can be obtained using a 3D multi-gradient echo sequence on a 3T scanner using imaging parameters TE/ATE/#TE/TR/FA/BW/FOV/matrix size=5 ms/5 ms/8/50 ms/20°/±62.50 kHz/21.8×24×12.8 cm³/232×256×64. Although example sequences and example parameters are described above, these are merely illustrative. In practice, other sequences and parameters can be used, depending on various factors (e.g., the size of region to be examined, a known or assumed range of susceptibility values of the subject, scan time considerations, device limitations, and so forth).

After acquiring MR data corresponding to a subject, the process 800 continues by determining a magnetic field based on the MR data (step 820). As noted above, in some cases, the MRI signal phase is affected by the magnetic field corresponding to the magnetic susceptibility distribution of the subject and the chemical shift of tissue components.

In some implementations as illustrated in FIG. 12a, the magnetic field can be determined from the complex MR data by fitting the detected signal as a sum over tissue spectral components, where each component signal characterized by an exponent with a negative real part representing signal decay and an imaginary part representing phase dependent on the magnetic field and the chemical shift (step 830). This fitting for such a complex signal model can be performed on iteratively using numerical optimization. A robust initialization for numerical optimization may be estimated using deep neural network. After determining the magnetic field based on the MR data, the process 800 continues by determining a relationship between the magnetic field and magnetic susceptibility (step 840). As described above, in some implementations, the relationship between the magnetic field and magnetic susceptibility can be expressed as a relationship between the magnetic field at a given location to the magnetic susceptibility at that location. In some cases, this relationship can be expressed in integral form, or in differential form. In the differential form, the relationship between the magnetic field at a given location to the magnetic susceptibility at that location can include an equation where a Laplacian of the magnetic field equals one third of the Laplacian of the magnetic susceptibility minus a second order derivative of the magnetic susceptibility.

In some implementations as illustrated in FIG. 12b, the magnetic field estimation step is bypassed, and the estimation of magnetic susceptibility map is performed directly on the multiecho complex data by forming a relationship between the multiecho complex data and the magnetic susceptibility (step 845).

Then process 800 continues by determining prior knowledge about tissue magnetic susceptibility distribution (step 850). One estimation of tissue susceptibility distribution is to use R2* values derived from the magnitude signal. High R2* values can be used to identify high susceptibility regions such as hemorrhages for preconditioning to accelerate the convergence of numerical optimization. An example of preconditioning is to separate the whole image volume into region with high susceptibility (including hemorrhages, air regions and background) and region with normal tissue susceptibility. The low (near zero) R2* regions can also be used for identifying regions of low susceptibility contrasts such as cerebrospinal fluid in the ventricles in the brain, oxygenated arterial blood in the aorta, or regions of pure fat in the abdominal wall. As water and fat have known susceptibility values, they can be used to serve zero references (to water) to generate absolute susceptibility values using a minimal variance regularization and to suppress streaking and shadowing artifacts.

After obtaining the susceptibility prior information and signal data noise property, the process 800 continues by estimating a magnetic susceptibility distribution of the subject based, at least in part, on the prior information and data noise property (step 860). As described above, estimating the susceptibility distribution of the subject can include determining a cost function corresponding to a susceptibility distribution, the magnetic field or the multiecho complex data, masks corresponding to regions of interest. The cost function in some includes a data fidelity term based on data noise property expressing the relationship between tissue susceptibility and the magnetic field in an integral or differential form or expressing the relationships between tissue susceptibility and the multiecho complex data, and a regularization term expressing prior information. The estimated susceptibility distribution of the subject can be determined by identifying a particular susceptibility distribution that minimizes one or more of these cost functions described above, in some cases, this can be determined numerically.

After estimating a magnetic susceptibility distribution of the subject, the process 800 continues by generating one or more images of the subject based on the estimated susceptibility distribution of the subject (step 870). These images can be electronically displayed on a suitable display device (e.g., an LCD display device, LED display device, CRT display device, or other display device that can be configured to show images) and/or physically displayed on a suitable medium (e.g., printed, etched, painted, imprinted, or otherwise physically presented on a sheet or paper, plastic, or other material).

In some cases, the estimated susceptibility distribution of the subject can be visualized using a color scale, where each of several colors is mapped to a particular susceptibility value or range of susceptibility values. Accordingly, a two dimensional or three dimension image can be generated, where each pixel or voxel of the image corresponds to a particular spatial location of the subject, and the color of that pixel of voxel depicts the susceptibility value of the subject at that location.

In some cases, the color scale can include a gradient of colors and/or a gradient of gray shades (e.g., a gray scale), in order to depict a range of susceptibility values. In some cases, for a color scale that includes a gradient of colors or a gradient of gray shades, one end of the gradient can correspond to the lowest susceptibility value in a particular window of susceptibility values (e.g., an arbitrarily selected window of values), and the other end of the gradient can correspond to the highest susceptibility value in the window of values. For instance, for gray scale that includes a gradient of gray shades between pure white and pure black, pure white can be used to indicate the highest susceptibility value in a particular arbitrary window of values, and pure black can be used to indicate the lowest susceptibility value in the window of values. Other relationships between a color/gray scale and susceptibility values is also possible. For example, in some cases, for gray scale that includes a gradient of gray shades between pure white and pure black, pure white can be used to indicate the lower susceptibility value in a particular arbitrary window of values, and pure black can be used to indicate the highest susceptibility value in the window of values. Although the examples are described in the context of gray scales, in practice, similar relationships between color scales and susceptibility values are also possible.

Susceptibility values and colors can be mapped linearly (e.g., such that each absolute change in susceptibility value corresponds to a proportional change in color), logarithmically (e.g., such that each exponential change in susceptibility value correspond to a linear change in color), or according to any other mapping. Although examples mapping between color scales and susceptibility values are described above, these are merely illustrative examples. In practice, other mappings are also possible, depending on the implementation.

Implementations of the above described techniques can be performed using a computer system. FIG. 13 is a block diagram of an example computer system 900 that can be used, for example, to perform implementations of the process 800. In some implementations, the computer system 900 can be communicatively connected to another computer system (e.g., another computer system 900), such that it receives data (e.g., MRI datasets), and analyzes the received data using one or more of the techniques described above.

The system 900 includes a processor 910, a memory 920, a storage device 930, and an input/output device 940. Each of the components 910, 920, 930, and 940 can be interconnected, for example, using a system bus 950. The processor 910 is capable of processing instructions for execution within the system 900. In some implementations, the processor 910 is a single-threaded processor. In some implementations, the processor 910 is a multi-threaded processor. In some implementations, the processor 910 involves a graphic processing unit. In some implementations, the processor 910 is a quantum computer. The processor 910 is capable of processing instructions stored in the memory 920 or on the storage device 930. The processor 910 may execute operations such as performing one or more of the techniques described above.

The memory 920 stores information within the system 900. In some implementations, the memory 920 is a non-transitory computer-readable medium. In some implementations, the memory 920 is a volatile memory unit. In some implementations, the memory 920 is a non-volatile memory unit.

The storage device 930 is capable of providing mass storage for the system 900. In some implementations, the storage device 930 is a non-transitory computer-readable medium. In various different implementations, the storage device 930 can include, for example, a hard disk device, an optical disk device, a solid-state drive, a flash drive, magnetic tape, or some other large capacity storage device. In some implementations, the storage device 930 may be a cloud storage device, e.g., a logical storage device including multiple physical storage devices distributed on a network and accessed using a network. In some examples, the storage device may store long-term data. The input/output device 940 provides input/output operations for the system 900. In some implementations, the input/output device 940 can include one or more of network interface devices, e.g., an Ethernet card, a serial communication device, e.g., an RS-232 port, and/or a wireless interface device, e.g., an 802.11 card (e.g. 802.11ax), a 3G wireless modem, a 4G wireless modem, a 5G wireless modem, etc. A network interface device allows the system 900 to communicate, for example, transmit and receive data. In some implementations, the input/output device can include driver devices configured to receive input data and send output data to other input/output devices, e.g., a keyboard, a mouse, a printer, a sensor (e.g., a sensor that measures component or system-related properties, a sensor that measures environmental-related properties, or other types of sensors), and a display device 960. In some implementations, mobile computing devices, mobile communication devices, and other devices can be used.

A computing system can be realized by instructions that upon execution cause one or more processing devices to carry out the processes and functions described above, for example, storing, maintaining, and displaying information. Such instructions can include, for example, interpreted instructions such as script instructions, or executable code, or other instructions stored in a computer readable medium. A computing system can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

Although an example processing system has been described in FIG. 13, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as performing one or more of the above described processes, can be implemented as one or more computer program products, e.g., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “processing module” may encompass all apparatus, devices, and machines for processing data, including by way of example a programmable processor, a computer, or multiple processors or computers. A processing module can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., erasable programmable read-only memory (EPROM), electrically erasable programmable read-only memory (EEPROM), and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. Sometimes a server is a general purpose computer, and sometimes it is a custom-tailored special purpose electronic device, and sometimes it is a combination of these things. Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

### Experimental Results.

We present some experimental implementations and results, exemplifying inventions disclosed in the above, particularly related to Eqs. 22-25.

We train a deep neural network to estimate perfusion parameters: perfusion F, permeability K^{trans}, capillary blood volume Vₚ and extravascular extracellular space volume Vₑ from time resolved 4D contrast enhanced images, such as DCE MRI images. In order to generate training data, a set of vasculatures containing arteries, capillaries and veins were generated based on constrained constructive optimization (CCO) method. Perfusion parameter maps were assumed to be a mixed Gaussian distribution. Tracer propagation inside vasculature and extravascular space were simulated using transport equation with parabolic flow assumption ignoring diffusion. Our test dataset included three parts: The first part is a set of concentration profile generated using the same rule as in training dataset. The second part is concentration profile in real tumor vasculature. The third part is human DCE MRI images. Details on training and testing data generation are described in the next section.

3D U-net was chosen as network architecture (FIG. 14). Concentration data at different time point were concatenated in feature dimension as input. The loss function was set as L1 norm of network output and ground truth of the parameters. The network weights were optimized using stochastic gradient decent method with epoch=40, learning rate=0.001, momentum=0.9.

We considered the vasculature and tracer propagation inside 64×64×64 mm³ volume. perfusion F, permeability K^{trans}, capillary blood volume Vₚ and extravascular extracellular space volume Vₑ were assumed to be a mixed Gaussian distribution:

$$F ( x ) = {\sum }_{i = 1}^{{N}_{0}} {e}^{- \frac{{( x - {x}_{i} )}^{2}}{{R}_{i}^{2}}} \qquad [ 31 ]$$

Here x=[x, y, z] is the 3D coordinate, N₀ is the number of Gaussian kernels and was set to 100. xᵢ is the center of kernels and Rᵢ is the radii of kernels, and was chosen from a uniform distribution between 2 mm to 10 mm. Kₜᵣₐₙₛ, Vₚ and Vₑ were calculated using the same method. Afterwards, F, K^{trans}, Vₚ and Vₑ were scaled to 0-120 mL/100 g/min, 0-0.1/min, 0.02-0.05 and 0.3-0.6, accordingly. One representative generated F, K^{trans}, Vₚ and Vₑ maps are shown in FIGS. 15c-15f. This parameter map generation method can be extended to parameters with different distribution and range easily to simulate flow and tracer propagation in different situations. After the parameter maps were determined, the arteries, capillaries and veins were built separately and combined together. The total volume was divided into 1×1×1 mm³ grids. The artery part was built using constrained constructive optimization (CCO) method. The construction flowchart is outlined as follows:

```
Do:
  1 Randomly draw a candidate terminal point P₁ inside the volume that
  satisfies criterion 1.
  If rand(1) > N₁:
    2 Find Nₐ vessel segments closest to P₁ and satisfies criterion 2.
    3 For each vessel segment Sᵢ found in step 2:
      3.1 The terminal point is given the flow F₁ from perfusion map F
      and connected to Sᵢ.
      3.2 Update the flow of vascular tree.
      3.3 Determine the optimal connecting point from P₁ to Sᵢ by
      minimizing the total vessel volume Vᵢ.
    4 P₁ is connected to the vessel segment Sⱼ that gives the minimal
    vessel volume Vⱼ
    5 Update the flow of vascular tree.
Else:
    2 P₁ is set as another arterial inlet.
    If node P₂ that satisfies criterion 3 can be drawn in the volume:
      3 P₂ was connected to P₁ as the first segment of this new artery and
      its flow was determined from perfusion map F.
    Else:
      3 Discard P₁.
    End
End
Until no more terminal point satisfies criterion 1.
```

Here, criterion 1 is satisfied if there is no other terminal inside the same grid P₁ belongs to. Criterion 2 is satisfied if the distance between vessel segment Sᵢ and P₁ is smaller than threshold d₁=5 mm. While minimizing the total vasculature volume, we assumed the cubic of vessel diameter is proportional to flow. The proportion is set to one as we only care relative vessel volume here. Venous vasculature was constructed separately using the same method.

Based on criterion 1, within each grid E with capillary vascular volume Vₚ(ε) there exists only one artery terminal and one vein terminal with the same flow F(ε). Capillaries were then constructed to connect each artery vein terminal pair. We assumed the capillaries stay inside the same grid as the terminal and the flow inside capillaries are plug flow, thus we only need to consider the length, radii and velocity of the capillaries instead of their morphology and bifurcation information. A random integer N₁ from a uniform distribution between 100 and 300 was chosen as the number of capillaries between the artery and vein terminal. Setting the radii, length and flow of the first capillary as a reference, N₁−1 random numbers from a uniform distribution between 0.8 and 1.2 were chosen as the relative radii to the first capillary. The same process was repeated to determine the relative length and flow to the first capillary. Next, we scaled the radii of the capillaries so that the average was 4.5 μm, scaled the flow velocity of the capillaries so that the total flow was F(ε), and scaled the length of the capillaries so that the total vascular volume equals to Vₚ(ε). In this way, a fully connected vascular network was constructed. As both arterial and venous vasculature are 1 direction tree while the flow of each terminal is known, the flow and velocity inside each vessel segment can be determined.

After the flow for each vessel segment was determined, tracer propagation in vascular network was simulated as follows: ignoring the diffusion inside vessel network, there is only axial direction velocity in vessel segment. In arterial we assumed a parabolic flow. Within one segment, the concentration c at each point can be expressed as:

$$c ( x , r , t ) = c ( 0 , r , t - \frac{x}{u ( r )} ) \qquad [ 32 ]$$

Here x is the distance of the point to the start of the cylinder assuming the velocity direction is positive direction, r is the distance of the point to the axis of the cylinder, u is the velocity. At the bifurcation point, we assume the concentration is preserved:

$${c}_{daughter} ( 0 , r , t ) = {c}_{father} ( {l}_{father} , \frac{{R}_{father}}{{R}_{daughter} r , t} ) \qquad [ 33 ]$$

In capillaries we assumed a plug flow. Apart from transport in axial direction, we also considered the exchange between capillaries and extravascular space. Each capillary was further divided into 10 elements. Within each element ξ inside voxel ε, the tracer concentration was simulated as follows:

$$\frac{\partial V ( ξ ) c ( ξ , t )}{\partial t} = F ( ξ - 1 ) c ( ξ - 1 , t ) - F ( ξ ) c ( ξ , t ) + {K}^{t r a n s} ( ε ) ( c ( ε , t ) - c ( ξ , t ) ) \qquad [ 34 ]$$

$$\frac{\partial {V}_{e} ( ε ) c ( ε , t )}{\partial t} = - {K}^{t r a n s} ( ε ) ( c ( ε , t ) - {\sum }_{ξ in ε} c ( ξ , t ) ) \qquad [ 35 ]$$

Here ξ−1 corresponds to the neighboring element at the upstream direction of element ξ. We assumed a uniform concentration distribution in extravascular space in each voxel ε. The size of voxel ε is set to 1 mm³ cube, consistent to the resolution of CCO method.

In venous vasculature, we again assumed a parabolic flow. While Eq. 32 still holds inside one vessel segment, at the meeting point, we assume the concentration of father branch is the average of concentration in daughter branch weighted by its flow:

$${c}_{father} ( 0 , r , t ) = {\sum }_{all daughter branch} \frac{{F}_{{daughter}_{i}}}{{F}_{father}} {c}_{{daughter}_{i}} ( {l}_{{daughter}_{i}} , \frac{{R}_{{daughter}_{i}}}{{R}_{father}} r , t ) \qquad [ 36 ]$$

In arterial side, father branch means the branch in upstream direction, while in venous side father branch means the branch in downstream direction. As long as flow is conserved at the connecting point, the concentration is also conserved. A concentration input

$$c ( t ) = 0.15 {e}^{- \frac{t}{45 s}} + 5.5 ( 1 - exp ( - \frac{t}{15 s} ) )$$

is used as the arterial input for the simulation, which is interpolated from the tracer concentration profile of hepatic artery in a DCE MRI scan. Eqs. 32-36 allow us to calculate the tracer concentration at each segment independently instead of updating all the segments at the same time.

In total, 1600 vasculatures and corresponding concentration profile were generated, 1200 of them were used as training data, 200 of them were used as validation and 200 of them were used as testing data.

Apart from the simulation on artificial vasculature, we also validated our deep learning method using simulation on real tumor vasculature and real DCE MRI images. For simulation based on real tumor vasculature, arteries and veins were segmented from optical projection tomography image of a rat glioma and were digitalized into one-dimension trees comprised of nodes and connections with certain radii and length. Limited by image resolution, more than 70% of the vessel segments are larger than 20 μm, thus the network lacks feeding arterioles and draining veins with diameter between 10 μm and 20 μm, and also capillaries with diameter smaller than 10 μm. Therefore, we constructed feeding arterioles, draining veins and capillaries using the same CCO method as described in previous section. F, K^{trans}, Vₚ and Vₑ maps were assumed to be a mixed Gaussian distribution as Eq. 1, and the tracer propagation was simulated based on Eqs. 2-6. Simulated concentration profile was down sampled to 1 mm voxel size and 5 s temporal resolution and used as the input for the neural network. Reconstructed parameter maps were then compared with the ground truth.

Six brain DCE MRI image of glioblastoma patients were acquired using a T1 fast-low angle shot, FLASH/vibe sequence. The scanning parameters were as follows: 40 dynamic acquisitions, 4.9 s per dynamic acquisition, TR=4.09 ms, TE=1.47 ms, flip angle=9°, phase=75%, bandwidth=400 Hz, thickness=4 mm, slice gap=0 mm, FOV=180*180 mm², matrix=192×144, TA=245 s. The contrast agent Gadodiamide (Omniscan, GE Medical Systems, Amersham, Ireland) was administered intravenously during the third dynamic acquisition using a power injector system (Spectris Solaris, MedRad, Indianola, PA, USA) at 0.1 mmol/kg body weight and 2 ml/sec, immediately followed by a 25-ml saline flush at a rate of 3.5 ml per second. As the input size of the neural network is 64×64×64, the DCE MRI images were interpolated to 1 mm voxel size and sliding window method with step size=3 voxels was applied to go over the whole volume.

Our proposed deep learning method can accurately reconstruct F, K^{trans}, Vₚ, and Vₑ from 4D tracer concentration profile. In test dataset 1 where the vasculature, parameter map and tracer concentration were constructed using the same method as training dataset, the relative root mean squared error (rRMSE) is smaller than 1% for F, K^{trans}, Vₚ and Vₑ (FIG. 16). In test dataset 2 where the vasculature was constructed based on real tumor vasculature, the rRMSE is 8% for F, 10% for K^{trans}, 11% for Vₚ and 9% Vₑ (FIG. 17).

Our deep learning method can be applied to DCE MRI processing to estimate perfusion parameters without requirement of arterial input function. FIG. 18 shows reconstructed F, K^{trans}, Vₚ and Vₑ map from a representative DCE MRI image of a glioblastoma tumor patient. A decent image quality was observed, the average F, K^{trans}, Vₚ, and Vₑ in tumor ROI are 62±21 mL/100 g/min, 0.03±0.01/min, 0.08±0.02 and 0.35±0.10, accordingly.

Certain features that are described above in the context of separate implementations can also be implemented in combination in a single implementation. Conversely, features that are described in the context of a single implementation can be implemented in multiple implementations separately or in any sub-combinations.

The order in which operations are performed as described above can be altered. In certain circumstances, multitasking and parallel processing may be advantageous. The separation of system components in the implementations described above should not be understood as requiring such separation.

A number of embodiments of the invention have been described. Nevertheless, it will be understood that various modifications may be made without departing from the spirit and scope of the disclosure. Accordingly, other embodiments are within the scope of the following claims.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for generating one or more images of an object, the method comprising:
obtaining complex magnetic resonance data collected by a magnetic resonance scanner, wherein the complex magnetic resonance data comprises magnitude and phase information regarding the object;
estimating a magnetic susceptibility distribution of the object based on the obtained complex magnetic resonance data, wherein estimating the magnetic susceptibility distribution of the object comprises:
determining a cost function, the cost function including: a data fidelity term involving modeling a magnetic susceptibility effect on the obtained complex magnetic resonance data; and a regularization term involving a first region with a low feature of susceptibility contrasts and a second region with a feature of susceptibility contrasts different from the feature of susceptibility contrasts of the first region;
minimizing the cost function; and
determining the magnetic susceptibility distribution of the object based on minimizing the cost function;
generating the one or more images of the object based on the determined magnetic susceptibility distribution of the object; and
presenting, on a display device, the one or more images of the object generated based on the determined magnetic susceptibility distribution.

2. The method of claim 1, wherein the regularization term involves one or more additional regions with features of susceptibility contrasts differing from features of susceptibility contrasts of the first and second regions.

3. The method of claim 1, wherein the feature of susceptibility contrasts of the first region is lower than the features of susceptibility contrast of the second region.

4. The method of claim 1, wherein the regularization term involves a penalty on a variation of susceptibility values in a region.

5. The method of claim 4, wherein the penalty varies with the feature of susceptibility contrasts of a region.

6. The method of claim 1, wherein the feature of susceptibility contrasts of a region involves decay rates of magnitude signals in voxels of the region.

7. The method of claim 1, wherein a region is formed by binning a group of voxels sharing a characteristic.

8. The method of claim 7, wherein the characteristic is a distribution of decay rates.

9. The method of claim 8, wherein the distribution is a rectangular or Gaussian function.

10. The method of claim 7, wherein the characteristic is a distribution in space.

11. The method of claim 1, wherein minimizing the cost function is obtained using a preconditioning method.

12. The method of claim 1, wherein the regularization term is implicitly formed, and wherein estimating the magnetic susceptibility distribution is realized using a deep neural network.

13. The method of claim 12, wherein the deep neural network is trained with labeled data or unlabeled data, or is untrained.

14. The method of claim 12, wherein the deep neural network is trained with network weights updated with test data.

15. The method of claim 1, wherein modeling the magnetic susceptibility effect on the obtained complex magnetic resonance data involves a comparison between the obtained complex magnetic resonance data and a susceptibility modeled complex signal at each echo time.

16. The method of claim 15, wherein the susceptibility modeled complex signal includes a phase involving echo time and magnetic susceptibility dipole convolution.

17. The method of claim 15, wherein the comparison comprises an Lp norm of the difference between the obtained complex magnetic resonance data and a susceptibility modeled complex signal at each echo time.

18. The method of claim 1, wherein modeling the magnetic susceptibility effect on the obtained complex magnetic resonance data comprises:
calculating a magnetic field experienced by tissue in the object from the obtained complex magnetic resonance data;
calculating a phase at each echo time according to the calculated magnetic field and a multiplicative scaling factor;
performing a comparison involving the calculated phase with a phase of the magnetic susceptibility dipole convolution at each echo time; and
dividing the determined magnetic susceptibility distribution by the multiplicative scaling factor.

19. The method of claim 18, wherein the comparison involving the calculated phase with the phase of the magnetic susceptibility dipole convolution at each echo time comprises calculating an Lp norm of a weighted difference between an exponential factor of the calculated phase and an exponential factor of the phase of the magnetic susceptibility dipole convolution at each echo time.

20-47. (canceled)

48. A system, the system comprising a processor and a non-transitory computer-readable medium having processor-executable instructions stored thereon for generating one or more images of an object, wherein the processor-executable instructions, when executed by the processor, facilitate:
obtaining complex magnetic resonance imaging data collected by a magnetic resonance scanner, wherein the complex magnetic resonance imaging data comprises magnitude and phase information regarding the object;
estimating a magnetic susceptibility distribution of the object based on the obtained complex magnetic resonance data, wherein estimating the magnetic susceptibility distribution of the object comprises:
determining a cost function, the cost function including: a data fidelity term involving modeling a magnetic susceptibility effect on the obtained complex magnetic resonance data; and a regularization term involving a first region with a low feature of susceptibility contrasts and a second region with a feature of susceptibility contrasts different from the feature of susceptibility contrasts of the first region;
minimizing the cost function; and
determining the magnetic susceptibility distribution of the object based on minimizing the cost function;
generating the one or more images of the object based on the determined magnetic susceptibility distribution of the object; and
presenting, on a display device, the one or more images of the object.

49-51. (canceled)
